(19) United States
(12) Patent Application Publication
(10) Pub. No.: US 2018/0231524 A1
(43) Pub. Date: Aug. 16, 2018

(54) IN VITRO METHODS OF IDENTIFYING MODULATORS OF NEUROMUSCULAR JUNCTION ACTIVITY

(71) Applicant: MEMORIAL SLOAN-KETTERING CANCER CENTER, New York, NY (US)

(72) Inventors: Lorenz Studer, New York, NY (US); Julius Steinbeck, Feldafing (DE)

(73) Assignee: MEMORIAL SLOAN-KETTERING CANCER CENTER, New York, NY (US)

(21) Appl. No.: 15/946,905

(22) Filed: Apr. 6, 2018

Related U.S. Application Data
(63) Continuation of application No. PCT/US2016/056113, filed on Oct. 7, 2016.
(60) Provisional application No. 62/238,531, filed on Oct. 7, 2015.

(51) Int. Cl. G01N 33/50; C12N 5/0793; C12N 5/074; C12N 5/077

(52) U.S. Cl. CPC G01N 33/5032; C12N 5/0619; C12N 5/0607; C12N 5/0658; G01N 33/5061; C12N 2506/02; C12N 2501/15; C12N 2501/155; C12N 2501/16; C12N 2501/41; C12N 2502/1335; G01N 33/5058

(57) ABSTRACT

The present invention relates to an in vitro human neuromuscular junction model prepared from a co-culture of human pluripotent stem cell (PSC)-derived spinal motorneurons and human myoblast-derived skeletal muscle cells. The present invention also provides for methods of screening compounds for their ability to modulate neuromuscular junction activity by determining whether a candidate compound increases or decreases the activity of the in vitro human neuromuscular junction model.

### CROSS REFERENCE TO RELATED APPLICATIONS

This application is a Continuation of International Patent Application No. PCT/US16/56113, filed Oct. 7, 2016, which claims priority to U.S. Provisional Application No. 62/238,531 filed Oct. 7, 2015, the contents of each of which are hereby incorporated by reference in their entirety herein, and to each of which priority is claimed.

### GRANT INFORMATION

This invention was made with government support under grant number NS052671 awarded by the National Institutes of Health and National Institute of Neurological Disorders and Stroke. The government has certain rights in the invention.

### 1. INTRODUCTION

The present invention relates to methods of identifying modulators of neuromuscular and/or muscular activity using an in vitro model of the human neuromuscular junction.

### 2. BACKGROUND OF THE INVENTION

Neuromuscular diseases are diseases which lead to impairment of motorneuron and/or muscle function due to the loss of motorneuron or muscle cells, reduction of motorneuron or muscle cell function, or degenerative changes in the motor pathways of the central (CNS) or peripheral (PNS) nervous systems. Such diseases are different from other neurodegenerative diseases such as Alzheimer's disease, which are caused by the destruction of neurons other than motorneurons. Typically, neuromuscular diseases are developmental or progressive, degenerative disorders. Symptoms may include difficulty swallowing, limb weakness, slurred speech, impaired gait, facial weakness and muscle cramps. Respiration may be affected in the later stages of these diseases, frequently resulting in death. The causes of most neuromuscular diseases are not known, but environmental, toxic, viral or genetic factors are all suspects.

The connection between spinal motorneurons and skeletal muscle is the crucial final pathway of the human pyramidal motor system controlling voluntary movements (Barker et al., 1985). It is severely affected in many traumatic, degenerative and inflammatory diseases, which are classically believed to affect mainly either the neuronal (Kuwabara and Yuki, 2013; Sendtner, 2014; Silva et al., 2014; Titulaer et al., 2011), or the muscle side (Mercuri and Muntoni, 2013; Plomp et al., 2015) of the neuromuscular junction. It is clear that muscle denervation and re-innervation dramatically alter muscle physiology (Cisterna et al., 2014; Daube and Rubin, 2009). Vice versa, there is increasing evidence that muscle-dependent trophic, cell adhesion, and axon-guidance signals play an essential role in the formation and maintenance of the neuromuscular junction. Physiological activity such as exercise or pathological conditions such as amyotrophic lateral sclerosis (ALS) and other neuromuscular diseases greatly affect strength and function of the neuromuscular junction (Moloney et al., 2014). Similar to an animal model, a human system to study neuromuscular development and disease should comprise the main components of the neuromuscular junction including spinal motorneurons and skeletal muscle and be amenable to functional testing and manipulation.

Application of pluripotent stem cell (PSC)-derived neurons in regenerative medicine and disease modeling ideally requires their integration into complex functional human networks or tissues. For several CNS cell types this need has been addressed by the development of more integrative tissue engineering approaches where pluripotent cells are used to generate miniature three-dimensional model versions of human organs (Lancaster and Knoblich, 2014). Yet, one of the most important properties of neurons, namely their ability to form functional synapses and transmit information to appropriate downstream targets, remains largely unexplored in human organoids and other PSC-based model systems.

Although progress has been made in the generation of spinal motorneurons from human PSCs (Amoroso et al., 2013; Calder et al., 2015; Chan et al., 2007; Davis-Dusenbery et al., 2014; Maury et al., 2015; Patani et al., 2011), their ability to functionally connect to and control human skeletal muscle function has not been assessed. Accordingly, there exists a need for an in vitro human model of the neuromuscular junction prepared from pluripotent stem cells that can be used to assess neuronal connectivity, and the effect of modulating compounds on such connectivity.

### 3. SUMMARY OF THE INVENTION

The present invention relates to a cultivated human neuromuscular junction (NMJ) prepared by cultivating human motorneurons and human muscle cells, for example where said motorneurons and optionally said muscle cells are products of in vitro differentiation.

In certain non-limiting embodiments, the present invention relates to an in vitro model of the human neuromuscular junction (NMJ), wherein the model is prepared by co-culturing human motorneurons with human muscle cells (e.g., myocytes) or muscle tissue.

In certain non-limiting embodiments, the human motorneurons are human pluripotent stem cell (PSC)-derived neurons. In certain non-limiting embodiments, the human muscle cells are human myoblast-derived skeletal muscle cells. In certain non-limiting embodiments, the human muscle cells are PSC-derived muscle cells.

In certain embodiments, the human PSC-derived spinal motorneurons are differentiated by contacting a human PSC with an effective amount of at least one Small Mothers Against Decapentaplegic (SMAD) inhibitor, at least one ventralizing factor, and at least one caudalizing factor.

In certain non-limiting embodiments, the at least one SMAD inhibitor is an inhibitor of Transforming growth factor β (TGFβ)/Activin-Nodal signaling, an inhibitor of bone morphogenetic proteins (BMP) signaling, or combinations thereof.

In certain non-limiting embodiments, the at least one ventralizing factor comprises an activator of the hedgehog pathway, for example, sonic hedgehog (SHH), purmorphamine, or combinations thereof.

In certain non-limiting embodiments, the at least one caudalizing factor is selected from the group consisting of retinoic acid (RA), a Wingless (Wnt) activating factor, and combinations thereof.

In certain non-limiting embodiments, the human muscle cells are obtained from a subject. In certain non-limiting embodiments, the muscle cells are de-differentiated into muscle cell precursors, for example, myoblasts, and cultured with the PSC-derived motorneurons.

In certain non-limiting embodiments, the NMJ model is prepared by co-culturing human motorneurons with human muscle tissue obtained from a subject.

In a non-limiting embodiment, the motorneurons of the in vitro model are under optogenetic control, wherein co-cultures of the motorneurons with muscle cells or tissue can be activated upon light stimulation to induce muscle movement.

In certain non-limiting embodiments, the PSC-derived motorneurons and/or muscle cells or tissue are prepared from PSCs obtained from a subject with a neuromuscular disease, for example, amyotrophic lateral sclerosis (ALS), myasthenia gravis and/or cachexia.

The present invention also relates to methods for identifying compounds that modulate NMJ activity through the use of the in vitro model of the human NMJ. In certain non-limiting embodiments, a candidate compound can be identified as an NMJ agonist through use of the in vitro NMJ model, wherein exposure of the NMJ to an effective amount of the candidate compound increases NMJ activity.

In certain non-limiting embodiments, a candidate compound can be identified as an NMJ antagonist through use of the in vitro NMJ model, wherein exposure of the NMJ to an effective concentration of the candidate compound decreases NMJ activity.

In certain non-limiting embodiments, the assay to identify modulators of NMJ activity measures the amplitude and/or frequency and/or duration of muscle contractions in the in vitro model as a measurement of NMJ activation, wherein an increase in the amplitude and/or frequency and/or duration of muscle contractions indicates an increase in NMJ activity, and a decrease in the amplitude and/or frequency and/or duration of muscle contractions indicates a decrease in NMJ activity.

In certain non-limiting embodiments, the assay measures the action potentials of the NMJ. In certain embodiments, the action potentials are measured in the motorneurons. In certain embodiments, the action potentials are measured in the muscle. In one non-limiting embodiment, an increase in amplitude and/or frequency and/or duration of action potentials indicates an increase in NMJ activity, and a decrease in amplitude and/or frequency and/or duration of action potentials indicates a decrease in NMJ activity.

In certain non-limiting embodiments, the assay measures the concentration or level of neurotransmitter released by the motorneurons, or present in the synapse between a motorneuron and muscle tissue, of the NMJ, wherein an increase in the concentration or level of neurotransmitter indicates an increase in NMJ activity, and a decrease in the concentration or level of neurotransmitter indicates a decrease in NMJ activity.

In certain non-limiting embodiments, the assay measures calcium current in the muscle and/or motorneuron in the NMJ model, wherein an increase in amplitude and/or frequency and/or duration of calcium current indicates an increase in NMJ activity, and a decrease in amplitude and/or frequency and/or duration of calcium current indicates a decrease in NMJ activity.

In certain non-limiting embodiments, the present invention provides for a method for identifying an agonist of neuromuscular junction activity comprising: (a) stimulating the motorneuron of the in vitro neuromuscular junction described herein in the presence of a candidate compound, and determining the activity of the in vitro neuromuscular junction; (b) stimulating the motorneuron of the in vitro neuromuscular junction described herein in the absence of the candidate compound, and determining the activity of the in vitro neuromuscular junction; (c) comparing the activity in (a) and (b); and (d) selecting the candidate compound as the agonist when the level of activity in (a) is greater than the level of activity in (b).

In certain non-limiting embodiments, the present invention provides for a method for identifying an antagonist of neuromuscular junction activity comprising: (a) stimulating the motorneuron of the in vitro neuromuscular junction described herein in the presence of a candidate compound, and determining the activity of the in vitro neuromuscular junction; (b) stimulating the motorneuron of the in vitro neuromuscular junction described herein in the absence of the candidate compound, and determining the activity of the in vitro neuromuscular junction; (c) comparing the activity in (a) and (b); and (d) selecting the candidate compound as an antagonist when the level of activity in (a) is less than the level of activity in (b).

The present invention also relates to methods for identifying genes that modulate NMJ activity through the use of the in vitro model of the human NMJ. In certain non-limiting embodiments, the activity of the NMJ can be assayed when the expression level of one or more genes expressed in a motorneuron and/or muscle of an NMJ, for example, a healthy wild-type NMJ, is decreased. In certain non-limiting embodiments, the activity of the NMJ can be assayed when the expression level of one or more genes expressed in a motorneuron and/or muscle of an NMJ, for example, a healthy wild-type NMJ, is increased. In certain non-limiting embodiments, the activity of the NMJ can be assayed when the expression level of one or more genes not normally expressed in a motorneuron and/or muscle of an NMJ, for example, a healthy wild-type NMJ, is expressed in the motorneuron or muscle. When an increase or decrease in expression level of a gene modulates NMJ activity, such a gene can be selected as an NMJ modulating gene.

The present invention also provides for kits comprising PSCs or PSC-derived motorneurons and skeletal muscle, or co-cultures thereof. In certain embodiments, the PSCs or PSC-derived neurons are human. In certain embodiments, the skeletal muscle is human myoblast-derived skeletal muscle. In certain embodiments, the skeletal muscle is PSC-derived muscle. In certain embodiments, the skeletal muscle is obtained from a subject.

### 4. BRIEF DESCRIPTION OF THE FIGURES

FIG. 1A-R. Optogenetic control in hPSC derived spinal motorneurons (MNs). (A) Shows a clonal hESC line carrying the hSyn-ChR2-EYFP transgene staining for OCT4 (POU5F1) and DAPI. (B) Shows that at day 20 (D20) MN clusters express ChR2-EYFP when examined under bright field (BF). (C) Shows that after purification of the neuronal clusters by sedimentation, MN clusters are enriched. (D) shows that after purification spinal motorneurons (sMN) markers are up-regulated, as measured by QRT-PCR. (E) Shows that after purification non-neuronal markers are down-regulated, as measured by QRT-PCR. (F) Shows that at day 30 of culture, spinal MNs express ChR2-EYFP and stain for HB9 and ISL1. (G) Shows that at day 30 of culture, spinal MNs co-stain for ChAT and SMI32. (H) Shows that differentiation of MNs by an alternative protocol (Maury et al., 2015) produced MNs expressing ChR2-EYFP+ MNs. (I) Shows that at day 30 of culture, spinal MNs (differentiated by an alternative protocol (Maury et al., 2015)) express ChR2-EYFP, HB9 and ISL1. (J) Shows that at day 60 of culture, spinal MNs (differentiated by an alternative protocol (Maury et al., 2015)) express ChR2-EYFP, ChAT and SMI32. (K) Shows a neuron in bright field and EYFP channel chosen for electrophysiology. (L) Shows that beyond day 60 (D60+) of culture, hESC-derived MNs fire action potentials in response to depolarizing current injection. (M, N) Show that mature ChR2+ hESC-derived MNs faithfully fire action potentials in response to optogenetic stimulation. (O) Shows a clonal hESC line carrying the hSyn-EYFP transgene staining for OCT4 and DAPI. (P) Shows that at day 30 of culture, purified spinal hESC-derived MNs express EYFP, HB9 and ISL1. (Q) Shows that mature EYFP+ hESC-derived MNs fires action potentials in response to current injection. (R) Shows that mature EYFP+ hESC-derived MNs do not respond to light stimulation. Scale bars 100 μM. Error bars represent SEM.

FIG. 2A-C. Generation of functional human myofibers. (A) Human myoblasts derived from an adult donor (hMA, upper panel) and a fetal donor (hMF, lower panel). (B) Human myofibers at day 17 of differentiation. (C) Calcium imaging in human myofibers on day 35. Acetylcholine (ACh) induces a robust calcium transient. Each trace resembles a distinct fiber. Scale bars 100 μM.

FIG. 3A-R. Characterization of neuromuscular co-cultures. (A, E) Co-cultures of spinal hESC-derived MNs with adult (hMA) and fetal (hMF) derived myofibers 1 week (1 W) after initiation, EYFP and bright field channels. (B, F) Co-cultures of spinal hESC-derived MNs with adult (hMA) and fetal (hMF) derived myofibers 6-8 weeks after initiation. (C, G) Quantification of muscle twitches in co-cultures in response to optogenetic stimulation for 50 s (upper panel) and 500 s (lower panel). Each trace resembles a distinct fiber. (D, H) Vecuronium (2 μM) blocks light-evoked contractility in adult (D) and fetal (H) myofibers. (I) EYFP and bright field picture of calcium imaging experiment shown in (J). (J) Ratiometric analysis of calcium transients in myofibers in response to optogenetic stimulation for 2 min (upper panel) and 40 min (lower panel). Each trace resembles a distinct fiber. (K) Sharp electrode recording from a single myofiber. Generation of vecuronium-sensitive action potentials in response to optogenetic stimulation at 0.2 and 2 Hz. (L) Long-term stability of neuromuscular connectivity. Movement in individual regions was quantified on day 5, 15 and 25 and normalized to movement on day 0. (M) Co-cultures contain a dense layer of vimentin+ and GFAP+ stroma. (N) Co-cultures show dense network of EYFP+ axons and desmin+ muscle fibers. (O) Multinucleated and striated myofiber in close contact with EYFP+ neuronal processes in contractile region. (P) High-power confocal imaging of clustered acetylcholine receptor (BTX) in close association with EYFP+ neuronal process and synaptophysin labeling. (Q, R) Contracting regions (left) and non-contracting regions (right) were compared for AChR clustering. Quantification of BTX+ dots revealed a significant increase in contracting/innervated regions. *p<0.05. In C, D, G, and H one pixel corresponds to 0.5 μm. Scale bars 100 μm, except I, K 50 μm, and P, Q 25 μm.

FIG. 4A-Q. Myasthenia gravis disease modeling. (A, D) Kinetogram of mature, contracting co-cultures of spinal MNs with adult myofibers (hMA) before the addition of myasthenia gravis (MG) IgG (patient H) and complement (A) or control IgG and complement (D). (B, E) Same co-cultures as in A and D, on day 3 after the addition of myasthenia gravis (MG) IgG and complement (B) or control IgG and complement (E). (C, F) Same co-cultures as in B and E after the addition of pyridostigmine (PYR, 10 μM) on day 3. (G) Quantification of movement in cultures treated with MG IgG (patient #1 and 2) and complement or control IgG and complement on day 3 as % of day 0. (H) Quantification of movement in cultures treated with MG IgG (patient #1 & 2 combined) and complement before and after the addition of pyridostigmine on day 3. (I) Recovery of movement on day 4 and day 6 after wash out of MG IgG (patient #1 & 2 combined) and complement on day 3. (J) Quantification of movement in cultures treated with MG IgG (patient #1), control IgG and in untreated cultures, all without complement. (K, L) Bright field and EYFP images of functional MN co-cultures with adult muscle (hMA) treated with MG IgG (patient #1) and complement or control IgG and complement at 48 h in regions selected for calcium imaging. (M) Quantification of the calcium increase in response to optogenetic stimulation in MG and control cultures and after the addition of PYR. (N) Percentage of reactive fibers in response to optogenetic stimulation in MG and control cultures and after the addition of PYR. (O, P, Q) Immunocytochemistry (O, P) and quantification (Q) for the deposition of human complement C3c onto the neuromuscular junction co-labeled for EYFP and BTX 24 h after the addition of MG IgG (patient #1) or control IgG and complement. Areas in small boxes with dotted line are magnified in boxes with solid line. Scale bars 100 μm in K, L; 10 μm in O, P. In A-F one pixel corresponds to 0.5 μm. n.s.=not significant, *p<0.05, **p<0.01, ***p<0.001. All error bars represent SEM.

### 5. DETAILED DESCRIPTION OF THE INVENTION

The present invention relates to the generation of an in vitro model of the human neuromuscular junction (NMJ), wherein the NMJ is prepared by co-culturing human pluripotent stem cell (PSC)-derived spinal motorneurons with human myoblast-derived skeletal muscle, or PSC-derived muscle cells. In certain embodiments, the neurons are under optogenetic control, wherein activation of the neurons can be achieved by stimulation with light. As described herein, the in vitro model can be used for identifying modulators of NMJ activity, and thereby compounds that modulate motorneuron and/or muscle activity. In certain embodiments, the in vitro model is prepared from PSCs from subjects with a neuromuscular disease, such that compounds can be identified that can modulate the activity of the NMJ in the diseased state, wherein the identified compounds may be therapeutically effective in treating the neuromuscular disease.

For purposes of clarity of disclosure and not by way of limitation, the detailed description of the invention is divided into the following subsections:

(i) cultivated neuromuscular junction (NMJ); and

(ii) methods of identifying NMJ modulators.

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the terms “modulates” or “modifies” refers to an increase or decrease in the amount, quality or effect of a particular activity of a motorneuron and/or a muscle upon which a motorneuron forms a synapse. “Modulators,” as used herein, refer to any inhibitory or activating compounds identified using an in vitro and/or in vivo assays, e.g., agonists, antagonists, allosteric modulators and their homologs, including fragments, variants and mimetics.

“Inhibitors” or “antagonists,” as used herein, refer to modulating compounds that reduce, decrease, block, prevent, delay activation, inactivate, desensitize or down regulate the biological activity of a motorneuron and/or a muscle upon which a motorneuron forms a synapse. The term “antagonist” includes full, partial, and neutral antagonists as well as inverse agonists.

“Inducers,” “activators” or “agonists,” as used herein, refer to modulating compounds that increase, induce, stimulate, activate, facilitate, enhance activation, sensitize or upregulate the biological activity of a motorneuron and/or a muscle upon which a motorneuron forms a synapse. The term “agonist” includes full and partial agonists.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

An “effective amount” of a substance as that term is used herein is that amount sufficient to effect beneficial or desired results, including clinical results, and, as such, an “effective amount” depends upon the context in which it is being applied. In the context of administering a composition to modulate the activity of a NMJ, an effective amount of a composition is an amount sufficient to increase or decrease activity of the NMJ. For example, the increase or decrease can be a 1%, 5%, 10%, 20%, 30%, 40%, 50%, 60%, 70%, 80%, 90%, 95%, 98%, 99% or 100% increase or decrease in NMJ activity. An effective amount can be administered in one or more administrations.

As used herein, and as well understood in the art, a “muscle” or “muscle tissue” is a tissue comprising myocytes, wherein the myocytes are organized as myocyte fibers (i.e., myofibers) comprising myofilament protein to form the muscle tissue.

As used herein, the term “disease” refers to any condition or disorder that damages or interferes with the normal function of a cell, tissue, or organ.

### 5.1 Cultivated Neuromuscular Junction (NMJ)

The present invention relates to a cultivated human neuromuscular junction (NMJ) prepared by cultivating human motorneurons and human muscle cells, for example where said motorneurons and optionally said muscle cells are products of in vitro differentiation.

In certain non-limiting embodiments, the present invention provides for an in vitro model of the human neuromuscular junction (NMJ) system that may be used to evaluate putative compounds for their ability to modulate the activity of the NMJ. Said model system may be used to test the effect(s) of a compound of the invention on muscle activity, for example contractibility.

In certain non-limiting embodiments, the in vitro model is prepared by co-culturing a human pluripotent stem cell (PSC)-derived spinal motorneuron with a human muscle cell (e.g., a myocyte) or a PSC-derived muscle cell, or muscle tissue, for example, human myoblast- or PSC-derived skeletal muscle. In certain embodiments the cells are non-human cells, for example, PSCs and muscle cells from a non-human mammal.

In one non-limiting embodiment, the PSC is an embryonic stem cell (ESC). In certain non-limiting embodiments, the PSC is an induced PSC (iPSC). Differentiation of the PSCs into spinal motorneurons can be achieved by contacting the PSC with at least one SMAD inhibitor, and in certain non-limiting embodiments, at least two SMAD inhibitors (for example, as described by Chambers et al., 2009, which is incorporated herein by reference in its entirety), at least one ventralization factor, for example, an activator of the hedgehog pathway (HH) (e.g., by administering sonic hedgehog (SHH) or purmorphamine), and at least one caudalization factor, for example, retinoic acid (RA) and/or a Wingless (Wnt) activating factor (for example, as described by Calder et al., J Neurosci. 2015 Aug. 19; 35(33):11462-81, which is incorporated herein by reference in its entirety).

In certain non-limiting embodiments, a SMAD inhibitor comprises an inhibitor of transforming growth factor beta (TGFβ)/Activin-Nodal signaling. In certain embodiments, the inhibitor of TGFβ/Activin-Nodal signaling neutralizes the ligands including TGFβs, bone morphogenetic proteins (BMPs), Nodal, and activins, or blocking their signal pathways through blocking the receptors and downstream effectors. Non-limiting examples of inhibitors of TGFβ/Activin-Nodal signaling are disclosed in WO/2010/096496, WO/2011/149762, WO/2013/067362, WO/2014/176606, WO/2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), which are incorporated by reference in their entireties herein for all purposes. In certain embodiments, the one or more inhibitor of TGFβ/Activin-Nodal signaling is a small molecule selected from the group consisting of SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

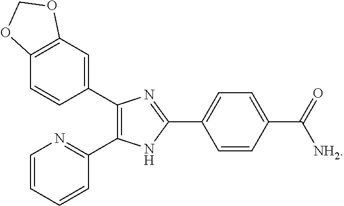 [SMILES: NC(=O)c1ccc(-c2nc(-c3ccc4c(c3)OCO4)c(-c3ccccn3)[nH]2)cc1]

In certain non-limiting embodiments, a SMAD inhibitor comprises an inhibitor of BMP signaling. Non-limiting examples of inhibitors of SMAD signaling are disclosed in WO2011/149762, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20, which are incorporated by reference in their entireties. In certain embodiments, the one or more inhibitor of BMP/SMAD signaling is a small molecule selected from the group consisting of LDN193189, derivatives thereof, and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula.

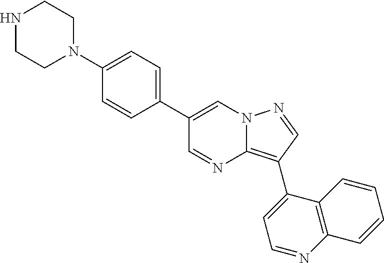 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference).

A presently disclosed differentiation method further comprises contacting the human stem cells with one or more activator of Wnt signaling. As used herein, the term “WNT” or “wingless” in reference to a ligand refers to a group of secreted proteins (i.e. Int1 (integration 1) in humans) capable of interacting with a WNT receptor, such as a receptor in the Frizzled and LRPDerailed/RYK receptor family. As used herein, the term “WNT” or “wingless” in reference to a signaling pathway refers to a signal pathway composed of Wnt family ligands and Wnt family receptors, such as Frizzled and LRPDerailed/RYK receptors, mediated with or without β-catenin. For the purposes described herein, a preferred WNT signaling pathway includes mediation by β-catenin, e.g., WNT/-catenin.

In certain embodiments, the one or more activator of Wnt signaling lowers GSK3β for activation of Wnt signaling. Thus, the activator of Wnt signaling can be a GSK3β inhibitor. A GSK3P inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 3β inhibitor” refers to a compound that inhibits a glycogen synthase kinase 3β enzyme, for example, see, Doble, et al., J Cell Sci. 2003; 116:1175-1186, which is incorporated by reference herein in its entirety.

Non-limiting examples of activators of Wnt signaling or GSK3β inhibitors are disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, which are incorporated by reference in their entireties. In certain embodiments, the one or more activator of Wnt signaling is a small molecule selected from the group consisting of CHIR99021, derivatives thereof, and mixtures thereof. “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino) ethylamino)nicotinonitrile with the following formula.

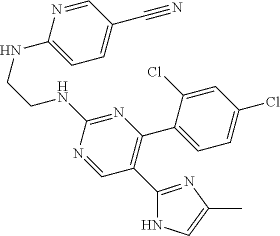 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3β and nanomolar IC50 values against rodent GSK3β homologs.

A presently disclosed differentiation method further comprises contacting the human stem cells with one or more activator of the hedgehog pathway (HH) (e.g., by administering Sonic hedgehog (SHH)). As used herein, the term “Sonic hedgehog,” “SHH,” or “Shh” refers to a protein that is one of at least three proteins in the mammalian signaling pathway family called hedgehog, another is desert hedgehog (DHH) wile a third is Indian hedgehog (IHH). Shh interacts with at least two transmembrane proteins by interacting with transmembrane molecules Patched (PTC) and Smoothened (SMO). Shh typically binds to PTC which then allows the activation of SMO as a signal transducer. In the absence of SHH, PTC typically inhibits SMO, which in turn activates a transcriptional repressor so transcription of certain genes does not occur. When Shh is present and binds to PTC, PTC cannot interfere with the functioning of SMO. With SMO uninhibited, certain proteins are able to enter the nucleus and act as transcription factors allowing certain genes to be activated (see, Gilbert, 2000 Developmental Biology (Sunderland, Mass., Sinauer Associates, Inc., Publishers). In certain embodiments, an activator of Sonic hedgehog (SHH) signaling refers to any molecule or compound that activates a SHH signaling pathway, including a molecule or compound that binds to PTC or a Smoothened agonist and the like. Non-limiting examples of activators of Wnt signaling or GSK3β inhibitors are disclosed in WO10/096496, WO13/067362, Chambers et al., Nat Biotechnol. 2009 March; 27(3):275-80, and Kriks et al., Nature. 2011 Nov. 6; 480(7378):547-51. Examples of such compounds are recombinant SHH, purified SHH, a protein Sonic hedgehog (SHH) C25II (i.e., a recombinant N-Terminal fragment of a full-length murine sonic hedgehog protein capable of binding to the SHH receptor for activating SHH, for example, R and D Systems catalog number: 464-5H-025/CF) and a small molecule Smoothened agonist such as, for example, purmorphamine.

In certain embodiments, the ventralization and caudalization factors are contacted with the cells in an effective amount from days 1-15 after the cells have been contacted with the at least one SMAD inhibitor. In one non-limiting embodiment, the ventralization and caudalization factors are contacted with the cells from days 1-20, 1-19, 1-18, 1-17, 1-16, 1-14, 1-13, 1-12, 1-11, or 1-10, and values in between, after the cells have been contacted with the at least one SMAD inhibitor. In certain non-limiting embodiments, the ventralization and caudalization factors are contacted with the cells beginning on at least day 0, 1, 2, 3, 4, 5, 6, 7, 8, 9, 10, 11, 12, 13, 14, 15, 16, 17, 18, 19 or 20 after the cells have been contacted with the at least one SMAD inhibitor, and are cultured with the cells until the cells are harvested and purified. In one non-limiting embodiment, the ventralization and caudalization factors are contacted with the cells for at least 10, 11, 12, 13, 14, 15, 16, 17, 18, 19, 20 or more days. Other methods of motorneuron differentiation known in the art can also be used, for example, as described by Maury et al., Nat Biotechnol. 2015 January; 33(1): 89-96 (Epub 2014 Nov. 10), which is incorporated by reference in its entirety herein.

In certain non-limiting embodiments the PSCs can be differentiated according to the methods described by U.S. Pat. No. 8,642,334; International Publication Nos. WO/2011/149762, WO/2013/067362, WO/2014/176606, and WO/2015/077648; and International Application No. PCT/US16/035312, filed Jun. 1, 2016, the contents of each of which are incorporated by reference in their entireties.

In certain non-limiting embodiments, the motorneuron is a recombinant cell expressing one or more proteins that enables optogenetic control of the motorneuron, for example, as described by Boyden et al., 2005; Zhang et al., 2011; Bryson et al., 2014; Cunningham et al, 2014; Steinbeck et al., 2015, each of which is incorporated by reference in their entireties herein. For example, stimulating a motorneuron expressing one or more of such proteins with light activates the motorneuron (e.g., by depolarizing the cell) such that the cell can activate the muscle tissue it synapses onto in the NMJ. In certain non-limiting embodiments, the one or more proteins can comprise a light-sensitive protein, derivatives thereof, and combinations thereof, for example, a light-gated ion channel such as a retinylidene protein (e.g., rhodopsins), for example, channelrhodopsins such as channelrhodopsin-1 or channelrhodopsin-2. Other examples of light-sensitive proteins include, but are not limited to, halorhodopsin, archaerhodopsin, bacteriorhodopsin, and proteorhodopsin.

In certain non-limiting embodiments, the light-sensitive protein is operably linked to a neuron specific promoter, for example, a synapsin promoter.

In certain non-limiting embodiments, the recombinant motorneuron can further express a detectable marker, such as, but not limited to, fluorescent proteins such as green fluorescent protein (GFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet, EYFP); β-galactosidase (LacZ); chloramphenicol acetyltransferase (cat); neomycin phosphotransferase (neo); enzymes such as oxidases and peroxidases; and/or antigenic molecules. In certain embodiments the detectable marker can be expressed as a fusion protein with a light-sensitive protein, for example, a channelrhodopsin-2-EYFP.

In one non-limiting embodiment, the PSC-derived motorneurons are purified after differentiation in culture for between about 10 and 15, 20, 25, 30, 35, 40, 45, 50 or more days, and values in between; or for at least about 10, 15, 20, 25, 30, 35, 40, 45, 50 or more days. In certain embodiments, the cells are purified by dissociation of the cultures (for example, on day 20) and sedimentation of the neuronal clusters, while the supernatant contains the non-neuronal cells.

In certain non-limiting embodiments, the PSC-derived motorneurons express detectable levels of one or more of homeobox gene 9 (HB9), neurofilament marker SMI32, Islet1 (ISL1), homeobox transcription factor NKX6.1, oligodendrocyte transcription factor 2 (OLIG2), choline acetyltransferase (ChAT), acetylcholine esterase (ACHE) and/or agrin (AG).

In one non-limiting embodiment, the PSCs and/or myoblasts described herein are derived from a subject that does not have neuromuscular disease. In certain non-limiting embodiments, the PSCs and/or myoblasts described herein are obtained from a subject that does have a neuromuscular disease, or at risk for having a neuromuscular disease, for example, ALS, myasthenia gravis, or cachexia. In certain non-limiting embodiments, the neuromuscular disease is primary lateral sclerosis (PLS), progressive muscular atrophy, progressive bulbar palsy, pseudobulbar palsy, spinal muscular atrophy (SMA), post-polio syndrome (PPS), spinal and bulbar muscular atrophy (SBMA), Charcot-Marie-Tooth disease (CMT), Guillain-Barré syndrome (GBS), or any other motor neuron disease known in the art.

In certain non-liming embodiments, the in vitro NMJ is used to model myasthenia gravis. In one non-limiting embodiment, the motorneuron and muscle components of the NMJ are co-cultured in the presence of immunoglobulin (e.g., IgG) from a myasthenia gravis patient, wherein the immunoglobulin comprises autoantibodies against proteins in the neuromuscular junction (e.g. the acetylcholine receptor, AChR) of the patient. In certain embodiments, the motorneuron and muscle are further co-cultured with active complement system components. In certain non-limiting embodiments, binding of the pathogenic antibody to AChR activates the complement cascade, resulting in destruction of the NMJ. In certain embodiments, the motorneuron and muscle are co-cultured in the presence of blood, serum, and/or plasma from a subject diagnosed with, or at risk of having, myasthenia gravis. In certain non-limiting embodiments, the in vitro NMJ model of myasthenia gravis is used in a method of screening for compounds that modulate NMJ activity, as described herein, for example, to identify compounds that increase activity of the NMJ.

In certain non-liming embodiments, the in vitro NMJ is used to model cachexia. In one non-limiting embodiment, the motorneuron and muscle components of the NMJ are co-cultured in the presence of condition media from a cancer cell culture. In one non-limiting embodiment, the motorneuron and muscle components of the NMJ are co-cultured in the presence of blood, serum, and/or plasma from a subject diagnosed with, or at risk of having, cachexia. In one non-limiting embodiment, the motorneuron and muscle components of the NMJ are co-cultured in the presence of proteolysis factors, and/or inflammatory cytokines, for example, but not limited to, tumor necrosis factor-alpha, interferon-gamma and interleukin-6. In certain non-limiting embodiments, the in vitro NMJ model of cachexia is used in a method of screening for compounds that modulate NMJ activity, as described herein, for example, to identify compounds that increase activity of the NMJ.

In certain non-limiting embodiments, the muscle component of the in vitro NMJ model is prepared from human primary myoblasts, or is derived from a PSC. In certain non-limiting embodiments, the muscle component of the in vitro NMJ model is prepared from human muscle cells that are de-differentiated into muscle cell precursors, for example, myoblasts, and cultured with the PSC-derived motorneurons. In certain non-limiting embodiments, the muscle component of the in vitro NMJ model is prepared from human muscle tissue obtained from a human subject. Any of the foregoing cells or tissue can be, for example, from an adult (hMA) and/or a fetal (hMF) donor subject.

In certain non-limiting embodiments, prior to differentiation, the human primary myoblasts, PSC, and/or human muscle cells that are de-differentiated into muscle cell precursors, can be cultured until a confluence level of at least about 40%, 45%, 50%, 55%, 60%, 65%, 70%, 75%, 80%, 85%, 90%, or more is achieved.

In certain embodiments, the human primary myoblasts, PSC, and/or human muscle cells that are de-differentiated into muscle cell precursors, are induced to differentiate into multinucleated myotubes, and then into myofibers, for between about 4 and 5, 6, 7, 8, 9, 10, 15, 17, 20, 25, 30, 35 or 40 days, and values in between, or for at least about 4, 5, 6, 7, 8, 9, 10, 15, 17, 20, 25, 30, 35, 40, 45, 50, 55, 60, 65, 70, 75, 80, 85, 90, 95 or more days. In certain non-limiting embodiments, following differentiation, the muscle tissue is responsive (e.g., contract) to stimulation with acetylcholine (ACh). In certain embodiments, the differentiated muscle tissue expresses detectable levels of ACh receptor (AChR) subunits, such as, for example, the fetal gamma subunit encoded by the CHRNG gene. In certain embodiments, the differentiated muscle tissue expresses detectable levels of muscle specific kinase (MuSK), desmin and/or myosin.

In one non-limiting embodiment, the human primary myoblasts, PSC, and/or human muscle cells that are de-differentiated into muscle cell precursors, are induced to differentiate when they reach 70% confluence, wherein the cells differentiate into multinucleated myotubes within about 4 to 7 days after the initiation of differentiation, and form myofibers by about days 10 to 17, wherein stimulation with acetylcholine (ACh, for example, 50 μM) can cause the myofibers to contract.

In certain non-limiting embodiments, the in vitro NMJ model is prepared from PSC-derived muscle cells.

In certain non-limiting embodiments, the PSC-derived motorneurons are co-cultured with the muscle cells or tissue described herein, for example, by culturing the motorneurons onto the muscle cells or tissue using methods known in the art.

In certain embodiments, the motorneurons used in the co-culture have been differentiated for between about 10 and 30 days, between about 10 and 25 days, between about 15 and 20 days, or between about 20 and 25 days, or for at least about 10, 15, 20, 25, 30, 35, 40, 45, 50 or more days, or for up to 10, 15, 20, 25, 30, 35, 40, 45, 50 or more days.

In certain embodiments, the muscle cells used in the co-culture have been differentiated for between about 4 and 25 days, between about 5 and 20 days, between about 5 and 15 days, between about 5 and 10 days, between about 10 and 17 days, or between about 4 and 7 days, or for at least about 4, 5, 6, 7, 8, 9, 10, 15, 17, 20, 25, 30, 35, 40, 45, 50, 55, 60, 65, 70, 75, 80, 85, 90, 95 or more days, or for up to 4, 5, 6, 7, 8, 9, 10, 15, 17, 20, 25, 30, 35, 40, 45, 50, 55, 60, 65, 70, 75, 80, 85, 90, 95 or more days.

In one non-limiting embodiment, the motorneurons used in the co-culture have been differentiated for between about 20 and 25 days, and the muscle cells used in the co-culture have been differentiated for between about 5 and 10 days.

To establish a neuromuscular co-culture, the PSC-derived motorneurons can be plated onto muscle cells or tissue, for example, myoblast- or PSC-derived muscle tissue, and then cultured under conditions sufficient for the motorneurons and muscle tissue to form functional neuromuscular junctions. In certain non-limiting embodiments, the motorneurons and muscle cells or tissue are co-cultured for a time sufficient for the growth of a layer of non-neuronal cells, for example, non-neuronal cells that hold the contracting muscle in place. In one non-limiting embodiment, the non-neuronal cells form connective tissue, for example, stromal cells that express vimentin and/or GFAP (Glial fibrillary acidic protein).

For example, the motorneurons and muscle tissue can be co-cultured for at least 4, 5, 6, 7, 8, 9, or 10 weeks or more to establish functional neuromuscular junctions. For example, following such co-culture, the muscle tissue exhibits a contractile response to stimulation with ACh. In embodiments wherein the motorneurons express a light-sensitive protein (i.e., are subject to optogenetic control), said muscle tissue exhibits a contractile response when the motorneurons are stimulated by light (e.g., a wavelength of light specific for the activation of the light-sensitive protein expressed by the motorneurons, such as 470 nm for excitation of Channelrhodopsin-2 (ChR2)).

### 5.2 Methods of Identifying NMJ Modulators

The present invention provides for methods of identifying compounds that modulate the activity of motorneurons and/or the muscle upon which the motorneurons form synaptic connections (i.e., modulation of NMJ activity). The capacity of a candidate compound to modulate the activity of a neuromuscular junction can be determined by assaying the candidate compound's ability to modulate the activity of an in vitro NMJ model, as described herein. Accordingly, the methods described herein provide a method for determining whether a candidate compound modulates any index of NMJ activity known in the art, for example, an increase or decrease in neurotransmitter release or stability; permeability to ions such as, for example, calcium, sodium or potassium; and/or connectivity between motorneurons and muscle. In one non-limiting embodiment, the candidate compound can modulate NMJ activity by increasing or decreasing neural connectivity between a motorneuron and muscle.

In certain non-limiting embodiments, the present invention provides for a method of identifying a candidate compound that modulates the activity of an NMJ by increasing the activity of a motorneuron and/or muscle of an in vitro NMJ model, wherein the candidate compound increases said activity by at least about 5%, 10%, 15%, 20%, 25%, 30%, 35%, 40%, 45%, 50%, 55%, 60%, 65%, 70%, 75%, 80%, 85%, 90%, 95%, 100%, or more, compared to the activity of the motorneuron and/or muscle when the candidate compound is not present. A candidate compound that modulates the activity of the NMJ by increasing NMJ activity can be selected as an NMJ agonist.

In certain non-limiting embodiments, the present invention provides for a method of identifying a candidate compound that reduces the activity of an NMJ by reducing the activity of a motorneuron and/or muscle of an in vitro NMJ model, wherein the candidate compound reduces said activity by at least about 5%, 10%, 15%, 20%, 25%, 30%, 35%, 40%, 45%, 50%, 55%, 60%, 65%, 70%, 75%, 80%, 85%, 90%, 95%, 100%, or more, compared to the activity of the motorneuron and/or muscle when the candidate compound is not present. A candidate compound that modulates the activity of the NMJ by reducing NMJ activity can be selected as an NMJ antagonist.

In certain non-limiting embodiments, a compound that modulates the activity of the NMJ by reducing NMJ activity can be used as an anesthetic and/or muscle relaxant, for example, as part of a therapeutic method of treatment.

In a particular non-limiting embodiment, the activity of an NMJ can be determined using an optogenetic technique. For example, a motorneuron of an NMJ can express a light-gated ion channel such as a retinylidene protein (e.g., a rhodopsin), for example, channelrhodopsins, which, when stimulated by light, depolarize the motorneuron such that an action potential is initiated. In certain embodiments, the motorneuron expresses Channelrhodopsin-2. In one embodiment, the Channelrhodopsin-2 is operably linked to a synapsin promoter. In certain non-limiting embodiments, the motorneuron expressing a light-gated ion channel can be stimulated with light focused on the motorneuron at a wavelength that activates the light-gated ion channel, wherein the activity of the motorneuron and/or muscle upon with the motorneuron synapses in the NMJ can be determined in the presence of a candidate compound.

In certain non-limiting embodiments, the motorneuron can be stimulated by injecting current, for example, depolarizing current, into the cell using techniques known in the art. In certain non-limiting embodiments, the motorneuron can be stimulated by depolarizing the cell's membrane potential, using techniques known in the art. In certain non-limiting embodiments, the muscle can be stimulated, for example, by contacting the muscle with a neurotransmitter. Upon stimulating the motorneuron and/or muscle, the activity of the NMJ can be determined in the presence and absence of a candidate compound.

In one non-limiting embodiment, the activity of an NMJ can be determined by measuring amplitude and/or frequency and/or duration of action potentials of the NMJ. In certain embodiments, the action potentials are measured in the motorneurons. In certain embodiments, the action potentials are measured in the muscle. The amplitude and/or frequency and/or duration of action potentials can be measured, for example, following stimulation of the motorneuron with a specific wavelength of light (e.g., when under optogenetic control), or when stimulated by injection of current, for example, depolarizing current, into the motorneuron, or upon direct stimulation of the muscle, for example, by contacting the muscle with a neurotransmitter. In one non-limiting embodiment, an increase in amplitude and/or frequency and/or duration of action potentials indicates an increase in NMJ activity, and a decrease in amplitude and/or frequency and/or duration of action potentials indicates a decrease in NMJ activity.

In certain non-limiting embodiments, the activity of an NMJ can be determined by measuring the level or concentration of neurotransmitter, for example, ACh, released by the NMJ motorneuron upon stimulation, wherein an increase in the concentration or level of neurotransmitter released by the motorneuron, or present in a synapse with a muscle in the NMJ, indicates an increase in NMJ activity, and a decrease in the concentration or level of neurotransmitter released by the motorneuron, or present in a synapse with a muscle in the NMJ, indicates a decrease in NMJ activity.

In certain non-limiting embodiments, the activity of an NMJ can be determined by measuring the amplitude and/or frequency and/or duration of the calcium current in the muscle and/or motorneuron of the NMJ, for example, upon stimulation of the motorneuron synapsing upon the muscle, or upon direct stimulation of the muscle, for example, by contacting the muscle with a neurotransmitter. In one embodiment, an increase in the amplitude and/or frequency and/or duration of the calcium current in the muscle and/or motorneuron indicates an increase in NMJ activity, and a decrease in the amplitude and/or frequency and/or duration of the calcium current in the muscle and/or motorneuron indicates a decrease in NMJ activity.

In certain non-limiting embodiments, the activity of an NMJ can be determined by measuring movement of the muscle of the NMJ upon stimulation of the motorneuron synapsing upon the muscle, or upon direct stimulation of the muscle, for example, by contacting the muscle with a neurotransmitter. In one embodiment, an increase in the amplitude and/or frequency and/or duration of muscle movement indicates an increase in NMJ activity and a decrease in the amplitude and/or frequency and/or duration of muscle movement indicates a decrease in NMJ activity.

In certain embodiments, a candidate compound can be identified as an NMJ agonist through use of the in vitro NMJ model described herein, wherein exposure of the NMJ to an effective amount of the candidate compound increases NMJ activity, for example, compared to an NMJ not contacted with the candidate compound.

In certain embodiments, a candidate compound can be identified as an NMJ antagonist through use of the in vitro NMJ model described herein, wherein exposure of the NMJ to an effective amount of the candidate compound decreases NMJ activity, for example, compared to an NMJ not contacted with the candidate compound.

In certain non-limiting embodiments, the NMJ comprises motorneurons expressing a light-gated ion channel, for example, Channelrhodopsin-2, and light-induced muscle contractions can be measured in functional co-cultures of the motorneurons with adult-derived (or fetal-derived) myoblasts before and after the incubation with IgG fractions (e.g., 200 nM total IgG) from a myasthenia gravis patient with elevated AChR antibody titers. Active complement can be added (e.g., added in the form of serum) together with the IgGs. Regions of the NMJ culture can be tested for NMJ activity prior to contacting the culture with the IgGs and complement, and again after IgG and complement exposure (e.g., at least three days after IgG and complement exposure).

In certain non-limiting embodiments, the present invention also provides for methods of identifying genes that modulate NMJ activity through the use of the in vitro model of the human NMJ. In certain non-limiting embodiments, the activity of the NMJ can be assayed when the expression level of one or more genes expressed in a motorneuron and/or muscle of an NMJ, for example, a healthy wild-type NMJ, is decreased. The expression level of the one or more genes can be decreased by contacting the motorneuron and/or muscle with, for example, an anti sense RNA, siRNA, or RNAi molecule targeted to an mRNA of the one or more genes; antibody, or active fragment thereof, that specifically binds to a protein expressed by the one or more genes; by introducing a mutation into the one or more genes that decreases the expression of a functional protein from the gene; or any other method known in the art for decreasing gene expression.

In certain non-limiting embodiments, the activity of the NMJ can be assayed when the expression level of one or more genes expressed in a motorneuron and/or muscle of an NMJ, for example, a healthy wild-type NMJ, is increased. In certain non-limiting embodiments, the activity of the NMJ can be assayed when the expression level of one or more genes not normally expressed in a motorneuron and/or muscle of an NMJ, for example, a healthy wild-type NMJ, is expressed in the motorneuron and/or muscle. In certain non-limiting embodiments, the expression level of a gene can be increased by recombinantly introducing an expression vector comprising the gene into the motorneuron and/or muscle. In certain embodiments, protein expressed by the gene can be prepared in vitro, and then contacted directly to the motorneuron and/or muscle.

When an increase or decrease in expression level of a gene modulates NMJ activity, such a gene can be selected as an NMJ modulating gene.

### 6. EXAMPLES

The presently disclosed subject matter will be better understood by reference to the following Example, which is provided as exemplary of the invention, and not by way of limitation.

### 6.1 Example 1

### Modeling of Human Neuromuscular Disease using an In Vitro Neuromuscular Junction under Optogenetic Control

### Summary

Capturing the full potential of human pluripotent stem cell (PSC)-derived neurons in disease modeling and regenerative medicine requires their interrogation in complex functional systems. Here we show the establishment of optogenetic control in human PSC-derived spinal motorneurons. Co-cultures of these motorneurons with human myoblast-derived skeletal muscle can be triggered to twitch upon light stimulation. Physiological and imaging approaches were used to characterize the newly established, all-human neuromuscular junction. To model neuromuscular disease we incubated these co-cultures with IgG from myasthenia gravis patients and with active complement. Myasthenia gravis is an autoimmune disorder that selectively targets the neuromuscular junction. We observed a reversible reduction in the amplitude of muscle contractions representing a surrogate marker for the characteristic loss of muscle strength. The ability to recapitulate key aspects of disease and its symptomatic treatment indicate that our novel neuromuscular junction assay will have broad implications for modeling neuromuscular disease and regeneration.

### Results

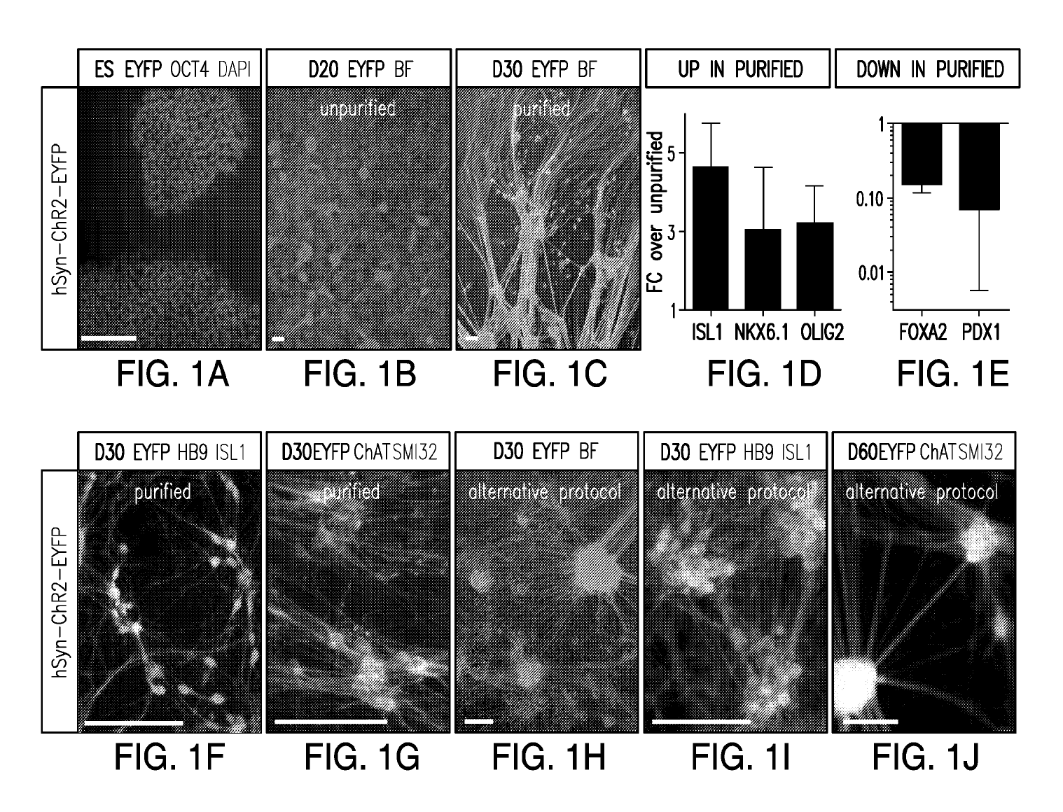

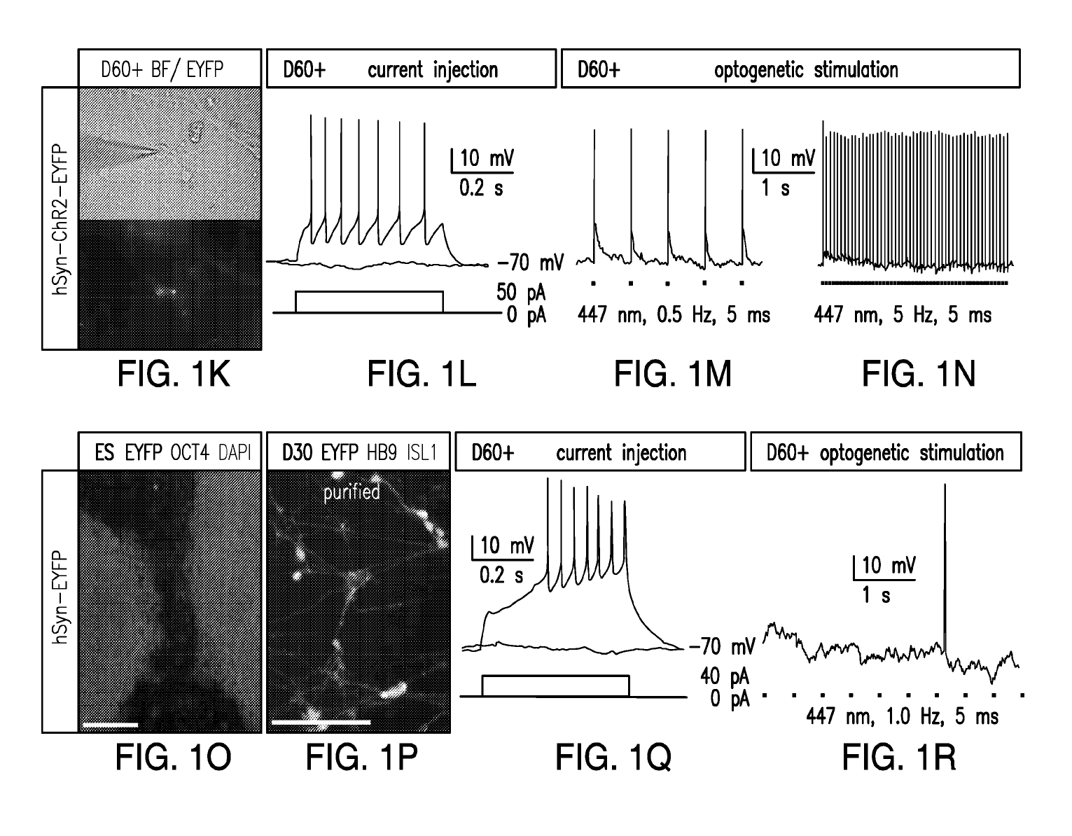

To establish optogenetic control in a human spinal MN population we transduced undifferentiated H9 hESCs with lentiviral vectors for the expression of Channelrhodopsin2-EYFP or EYFP alone under control of the human synapsin promoter. The synapsin promoter was selected for its faithful and robust expression in human PSC-derived neurons. Clonal hESC lines were expanded and validated by PCR for genomic integration of the transgenes (data not shown) as well as maintenance of pluripotency marker expression (FIG. 1A, O). Only ESC clones with robust transgene expression across various neuronal differentiation paradigms (Steinbeck et al., 2015) were selected for further experiments. Differentiation into spinal motorneurons was achieved by combining dual SMAD inhibition (Chambers et al., 2009) with activation of the hedgehog pathway for ventralization and exposure to retinoic acid for caudalization (Calder et al., 2015). By day 20 of differentiation the ChR2-EYFP transgene was expressed strongly in the neuronal clusters emerging under those culture conditions (FIG. 1B). We developed a simple purification procedure involving dissociation of the cultures on day 20 and sedimentation of the neuronal clusters while the supernatant, containing the non-neuronal cells, was discarded. This strategy allowed for a significant purification of hESC-derived MNs (FIG. 1C). QRT-PCR analysis of 5 consecutive MN differentiations confirmed a 3-fold enrichment of the bona-fide spinal motorneuron (sMN) markers ISL1, NKX6.1 and OLIG2 in purified MN cultures (FIG. 1D), whereas markers for non-neuronal contaminants (FOXA2, PDX1) were approximately 10-fold depleted in purified MN cultures (FIG. 1E). In an additional QRT-PCR experiment on purified MNs on day 40 we found the physiologically relevant MN markers choline acetyltransferase (ChAT) acetylcholine esterase (ACHE) and agrin (AG) to be expressed (CHAT 60.3±29.6% HPRT, ACHE 273.6±59.3% of HPRT, AG 79.3±32.1% of HPRT). By immunocytochemistry we confirmed that purified MNs expressed HB9 and ISL1 in combination with ChR2-EYFP (FIG. 1F) as well as ChAT and the mature neurofilament marker SMI32 (FIG. 1G). An alternative protocol for MN induction (Maury et al., 2015) produced comparable results (FIG. 1H-J). Optogenetic control was validated in electrophysiological experiments. In matured spinal motorneurons (beyond day 60), which were identified under bright field and green fluorescent optics (FIG. 1K), tonic action potential (AP) firing was evoked from a membrane potential held at −70±2 mV by injection of depolarizing current steps (FIG. 1L). Resting membrane potential was −62.4±4.8 mV. In addition, 4 out of 4 ChR2 expressing neurons fired light-induced APs over a broad frequency range form 0.2-10 Hz (FIG. 1M, N). Spike fidelity was 100% from 0.2 to 2 Hz, 93.3±5.7% at 5 Hz and 65.5±23.3% at 10 Hz. Purified neurons from the EYFP hESC line also expressed HB9 and ISL1 (FIG. 1O, P) and could be induced to fire APs by current injection (FIG. 1Q). Resting membrane potential was −58.4±2.6 mV. As expected, 2 out of 2 tested EYFP+ neurons did not respond to any light stimulation (FIG. 1R).

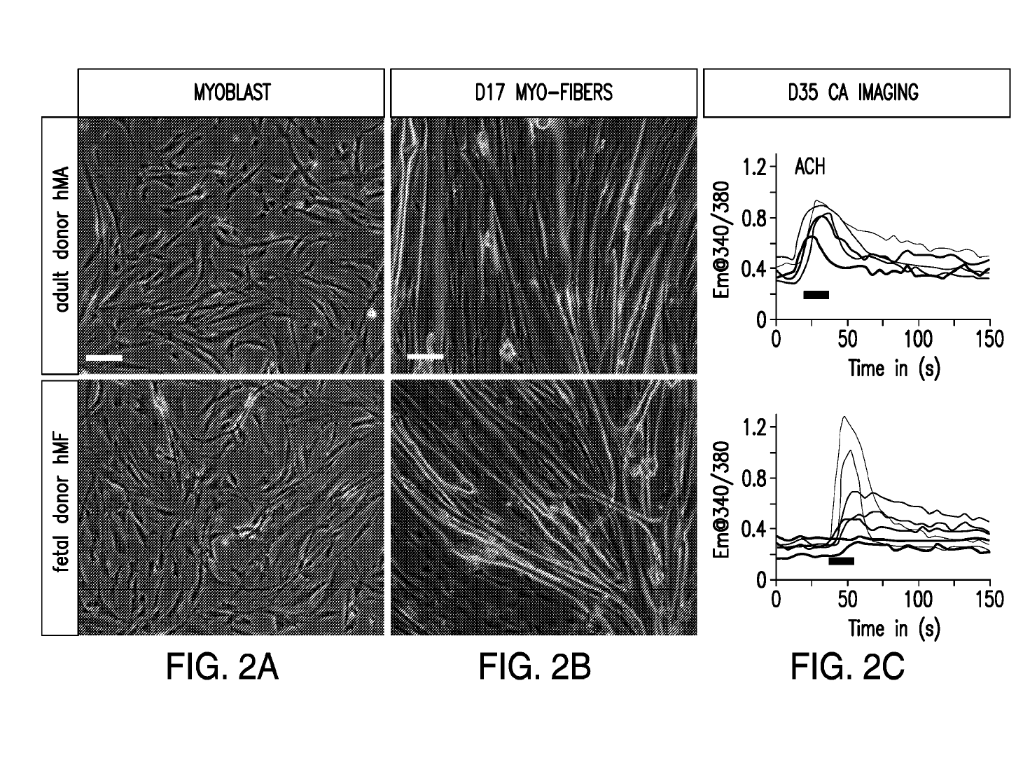

To obtain functional skeletal muscle in vitro, we used human primary myoblasts from an adult (hMA) and a fetal donor (hMF). Both types of myoblasts (FIG. 2A) were induced to differentiate when they reached 70% confluence. Both myoblast cultures fused to form multinucleated myotubes within 4 to 7 days after the initiation of differentiation. Stimulation with acetylcholine (ACh, 50 μM) caused adult and fetal myofibers to contract with increasing reliability from day 10 to day 17 (FIG. 2B). Muscle functionality was further assessed in calcium imaging experiments. Muscle cultures plated on glass coverslips were incubated with the calcium dye Fura2 on day 35 of differentiation. After stimulation with ACh the fibers generated a distinct calcium transient (FIG. 2C). Quantification of AChR subunits by QRT-PCR revealed that day 30 muscle cultures expressed the fetal gamma subunit (CHRNG, 32.2±8.6% of HPRT in hMA cultures and 97.1±33.4% of HPRT in hMF cultures) whereas the adult epsilon subunit was almost undetectable (CHRNE<0.2% of HPRT expression in hMA and IMF cultures, CHRNG:CHRNE ratio>100 for both muscle types). Human myo-cultures also expressed muscle specific kinase (MuSK, 44.8±18.5% of HPRT in hMA cultures and 73.8±15.8% of HPRT in hMF cultures). Immunocytochemical analysis confirmed that multinucleated myotubes expressed myosin, whereas the mesenchymal stroma expressed the intermediate filament vimentin. Structurally intact myotubes could be maintained in culture at least until day 90, but did not develop the typical skeletal muscle striation under these conditions.

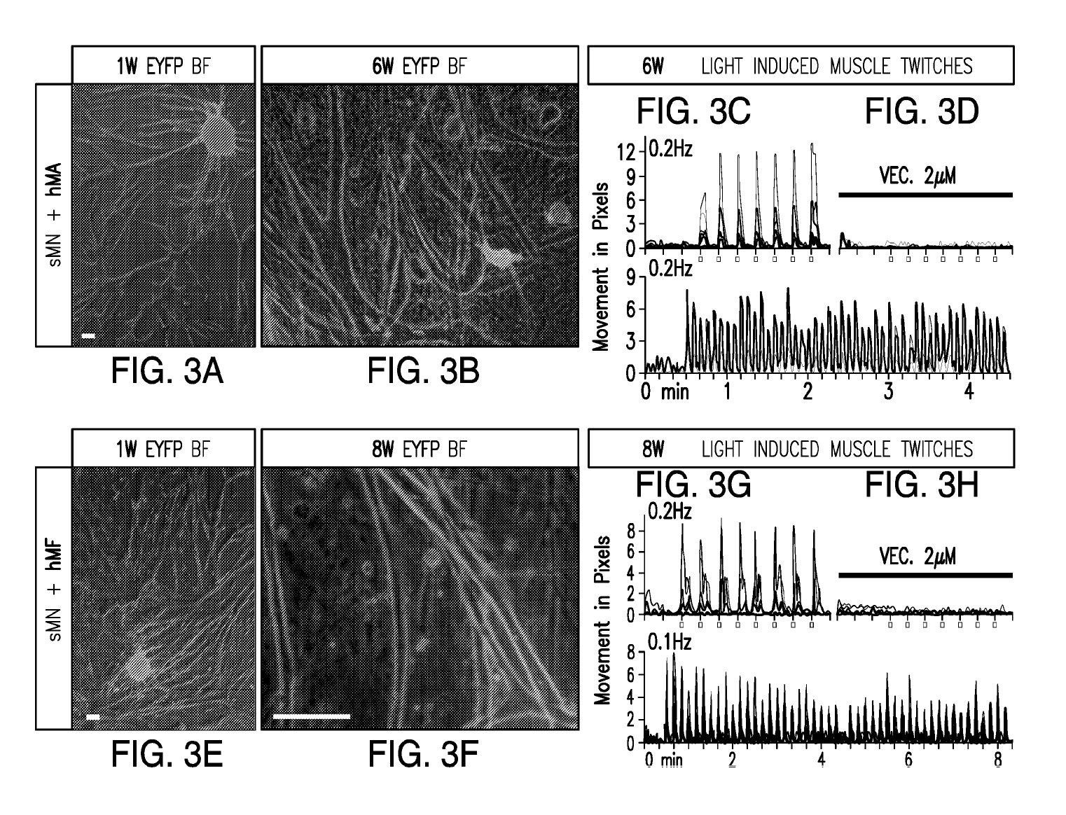

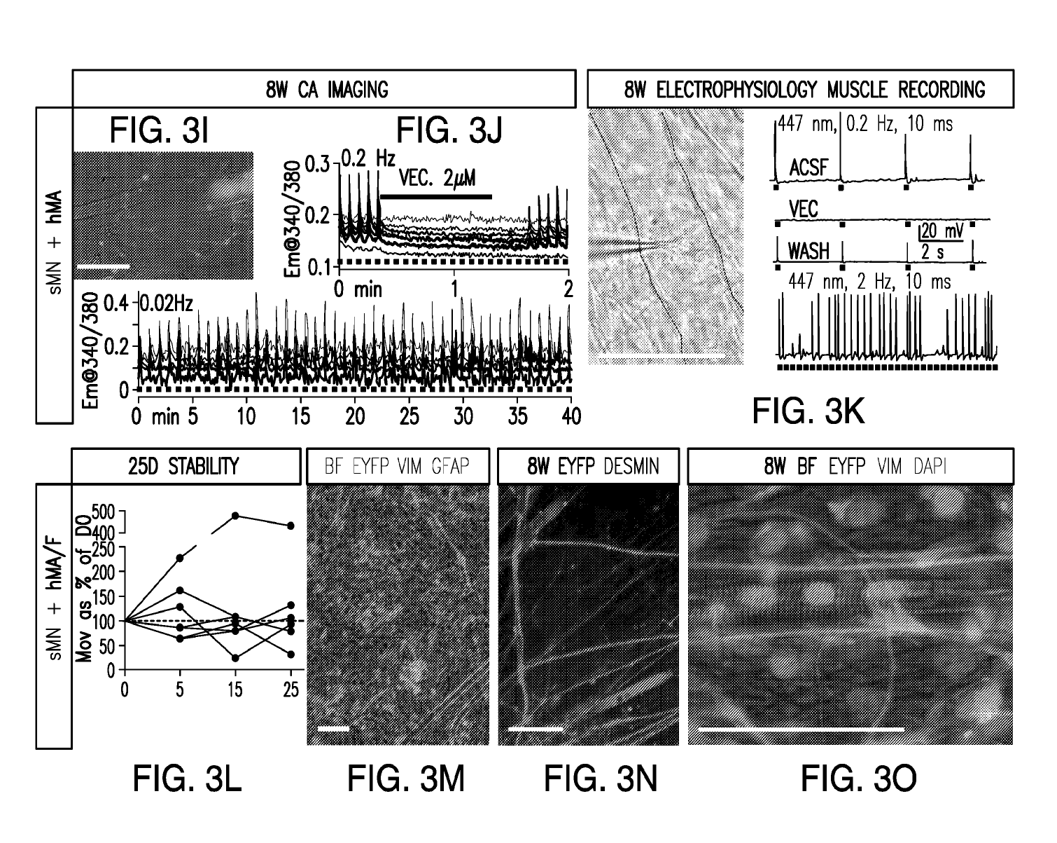

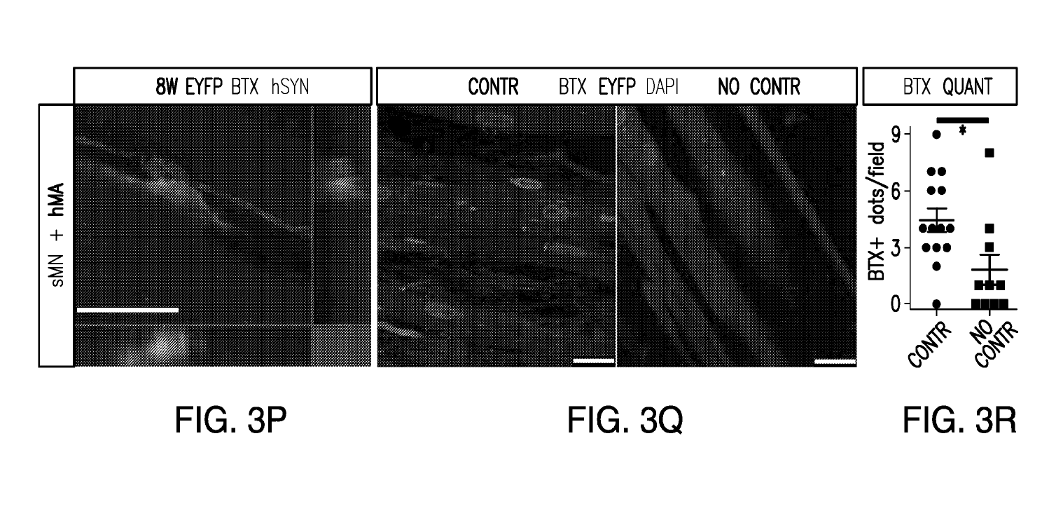

To establish the neuromuscular co-culture, we used the purified ChR2-expressing spinal motorneurons (day 20-25) and plated them onto pre-differentiated skeletal myofibers (day 5-10). After plating, hESC-derived spinal MN cell bodies mostly remained within the neuronal clusters but extended axons across the adult and fetal muscle (up to 2 mm within the first week, FIG. 3A, E). Co-cultures were tested for the establishment of neuromuscular connectivity in weekly intervals. For this purpose the cultures were observed under bright field illumination and intermittently stimulated with blue light pulses. Six to eight weeks after initiation of the co-culture, the elongated cylindrical myoblast-derived muscle fibers remained morphologically intact and started to contract in response optogenetic stimulation (470 nm, 0.2 Hz, 300 ms pulse width). FIGS. 3B, F show mature co-cultures with both muscle types. FIG. 3C, G show the quantification of muscle twitches of individual fibers from such cultures. The lower panels show long term experiments over 8.5 min (FIG. 3C at 0.2 Hz, FIG. 3G at 0.1 Hz). 630 nm light never caused any muscle contraction, indicating that muscle twitching was a result of ChR2 activation. Addition of vecuronium (2 μM), an antagonist of the nicotinic acetylcholine receptor (AChR) completely blocked the light induced muscle twitches in all tested cultures (FIG. 3D, H). These data indicate that connectivity was indeed established through a functional neuromuscular cholinergic synapse and not the result of cell fusion (i.e. ChR2 transfer into the muscle membrane which would cause direct muscle activation). Similar results were achieved when ChR2+ MNs generated through an alternative protocol (Maury et al., 2015) were plated onto hMA- or hMF-derived muscle (data not shown). Most MN-only cultures never showed any light induced twitching. However, approximately 20% of long term MN-only differentiations produced a non-neural overgrowth, which sometimes displayed light induced, vecuronium sensitive twitching. These data suggest that MN cultures with suboptimal purification may contain PSC-derived myogenic cells. The derivation of precursors capable of generating both spinal motorneurons and paraxial mesodermal structures including skeletal muscle have been reported recently (Gouti et al., 2014). However, such PSC-derived muscle-like cells never showed the isolated, elongated morphologies typical for primary myoblast-derived fibers,

We next characterized key physiologic parameters in the functional neuromuscular cultures. Matured co-cultures were incubated with the calcium dye Fura2 and mounted into an imaging chamber for continuous perfusion. In regions that were previously identified to show muscle contraction in response to light stimulation (FIG. 3I), 470 nm optogenetic stimulation produced calcium spikes in myofibers, which could be blocked by vecuronium (6/6 cultures, FIG. 3J). The lower panel of FIG. 3J confirms long-term stability of neuro-muscular excitability in a calcium imaging experiment over 45 minutes. In a separate study, skeletal myotubes (n=5), identified by their cylindrical and striated appearance under phase contrast microscopy and by their ability to undergo light-induced twitching were selected for intracellular recordings (FIG. 3K). Light-responsive myotubes were impaled with sharp electrodes and muscle APs were recorded during 447 nm optogenetic stimulation at frequencies from 0.2 to 2 Hz. Spike fidelity was 100% at 0.2 Hz, 93.3±6.7% at 0.5 Hz, 75±15.0% at 1 Hz and 80±10.0% at 2 Hz. Vecuronium (2 μM) completely blocked light-induced APs in myofibers in a reversible manner. To address the stability of neuromuscular connectivity over the course of several days and weeks, contractile regions (n=7) were assessed every 5 days. Quantitative analysis revealed that all 7 regions remained responsive to optogenetic stimulation and contraction increased to 137.3±50.7% until day 25 as compared to day 0 (FIG. 3L).

Morphological characterization of the co-cultures revealed the presence of a layer of non-neuronal cells, which are likely necessary to hold the contracting muscle in place. The majority of these stromal cells expressed vimentin and a minority GFAP (FIG. 1M). In most contractile regions a dense network of neuronal processes was found in close contact with myotubes staining for desmin (FIG. 3N) or myosin (data not shown). Neuronal EYFP+ boutons were found to be in close contact with striated, multinucleated muscle fibers (FIG. 3O). The acetylcholine receptor on myofibers was labeled with bungarotoxin (BTX). High power confocal imaging (FIG. 3P) revealed plaque-like clustering (Marques et al., 2000) of the acetylcholine receptor on muscle membranes in close apposition to MN synaptic terminals. Quantification of BTX+ dots on myofibers revealed a significant increase in contracting and strongly innervated regions as compared to non-contracting regions (FIG. 3Q, R; two-tailed unpaired t test, p=0.016, t=2.60). Quantification of AChR subunits by QRT-PCR revealed that co-cultures matured for 6 weeks expressed the fetal gamma subunit (CHRNG, 17.3±7.5% of HPRT in hMA co-cultures and 27.4±9.0% of HPRT in hMF co-cultures) whereas the adult epsilon subunit was almost undetectable (CHRNE<0.1% of HPRT expression in hMA and hMF co-cultures, CHRNG:CHRNE ratio >100 for both muscle types). Therefore, co-culturing with human spinal motorneurons did not induce expression of the adult epsilon subunit up until this time point. Neuromuscular co-cultures also expressed muscle specific kinase (MuSK, 7.9±1.9% of HPRT in hMA co-cultures and 27.7±10.3% of HPRT in IMF co-cultures).

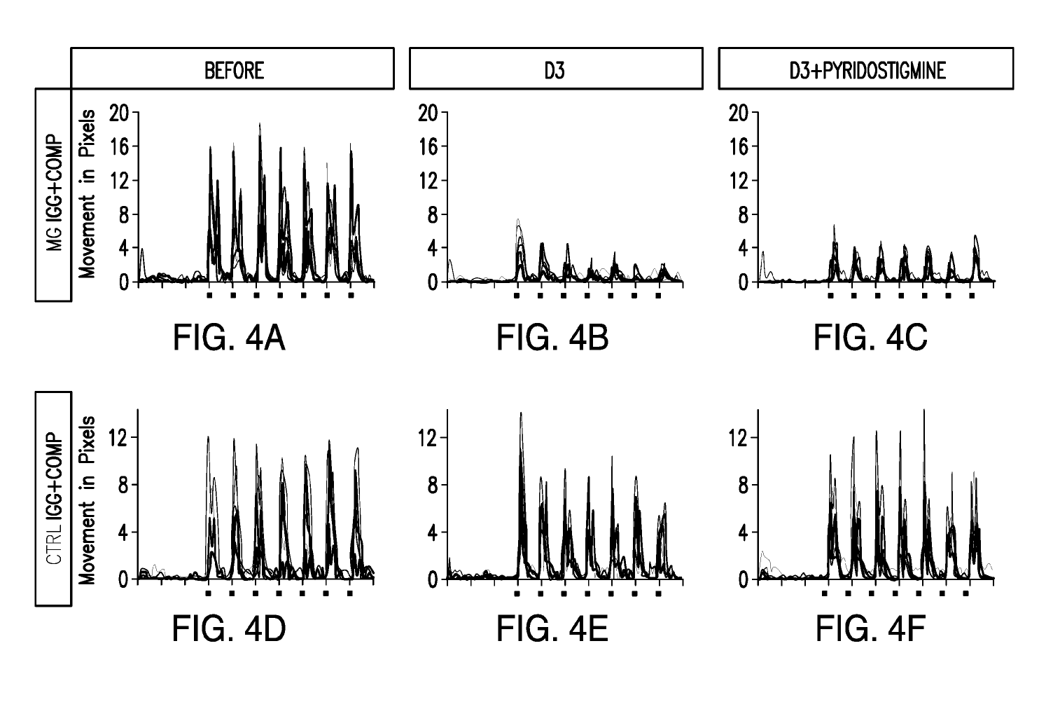

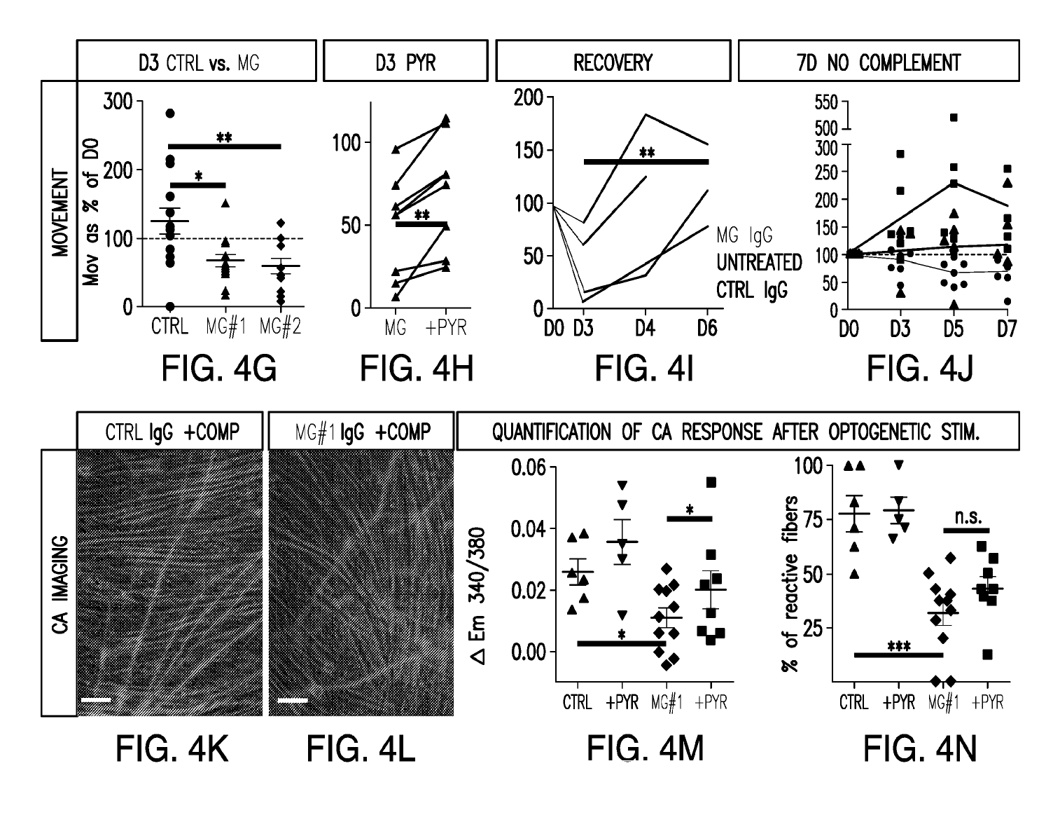

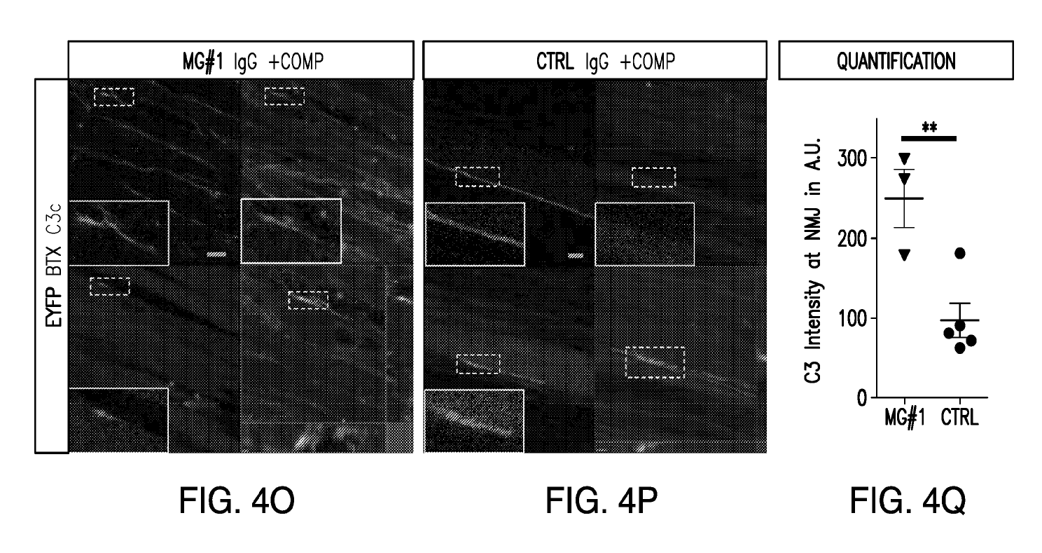

Next we sought to address whether the functional neuromuscular co-cultures were suitable to model a classic human neuromuscular disease. Myasthenia gravis (MG) (Toyka et al., 1977; Verschuuren et al., 2013) is caused by the emergence of autoantibodies against proteins in the neuromuscular junction (e.g. the acetylcholine receptor, AChR). Binding of the pathogenic antibody to AChR activates the complement cascade, resulting in destruction of the neuromuscular endplate (Sahashi et al., 1980), which ultimately causes progressive muscle weakness in patients. We therefore quantified light-induced muscle contractions in functional co-cultures of MNs with adult-derived myoblasts (hMA) before (FIG. 4A, D) and after the incubation with IgG fractions (200 nM total IgG) from two MG patients with clearly elevated AChR antibody titers (#1 and #2). Sandoglobulin (SG) polyvalent IgG served as control. 2% fresh human serum containing active complement was added together with all IgGs. When the exact same culture regions were tested again three days after IgG and complement exposure, we found that muscle twitches in response to light stimulation were reduced in cultures incubated with MG IgG and complement (FIG. 4B) but not in control cultures incubated with control IgG and complement (FIG. 4E). Careful quantification of muscle twitches in contractile cultures revealed that compared to the initial movement before addition of IgG and complement (day 0, 100%) control cultures (n=14) showed an increase in muscle contraction to 125% on day 3. In contrast, cultures incubated with IgG from either of the MG patients showed a significant decrease (#1, n=14, 68% and #2, n=11, 60%) in contractility (FIG. 4G, One-way ANOVA, p=0.0046, F (2,36)=6.25). Dunnett's multiple comparisons test revealed significant differences between the control group and either of the MG groups (CTRL vs. #1, p<0.05, q=2.93 and CTRL vs. #2, p<0.01, q=3.13), suggesting that a myasthenic phenotype had been introduced. To test if treatment responses could be modeled, we incubated MG cultures (FIG. 4C, F, n=8) and controls (FIG. 4F, n=4) with the ACh esterase inhibitor pyridostigmine (PYR, 10 μM). We found that PYR application in MG cultures induced a significant therapeutic effect (FIG. 4C and 4H, +22%, two-tailed paired t test, p=0.002, t=4.69). Next, we assessed if the myasthenic phenotype was reversible after washout of MG IgG and complement, mimicking plasmapheresis therapy (Gold et al., 2008). MG IgG and complement were added to the media on day 0 and identical culture regions (n=4) were tested again 1 and 3 days after wash out on day 3 (days 4 and 6, respectively). Quantitative analysis revealed that in 4 out of 4 cultures, wash out of MG IgG and complement resulted in the reversal of the myasthenic phenotype (FIG. 4I, D3 vs. D6, two-tailed paired t test, p=0.0098, t=10.09). We also tested the effect of control and MG#1 IgG without the addition of complement over a 7-day time course. Untreated cultures (n=5) and cultures treated with CTRL IgG (n=6) increased in contractility over time, whereas cultures treated with MG#1 IgG (n=7) showed a delayed but significant decrease in contractility to approximately 70% on day 5 and day 7 (CTRL vs. MG on day 7, Dunnett's multiple comparisons test, p<0.05, q=3.10).

To further characterize the myasthenic phenotype we performed calcium imaging experiments. Acute application of MG#1 IgG did not reduce the light induced calcium signal recorded from myofibers (0.2 to 5 μM, up to 60 min, data not shown). Therefore cultures were pretreated with control and MG#1 IgG and human complement for 2 days. Calcium imaging was performed in regions with similar amounts of muscle fibers and dense innervation (FIG. 4K, L). The light-induced calcium peaks, when quantified in all fibers identifiable in the visual field, were significantly weaker in cultures treated with MG#1 IgG and complement (n=11) as compared to controls (n=6, FIG. 4M, CTRL vs. MG, two-tailed unpaired t test, p=0.012, t=2.83). After application of PYR, we detected a small but significant increase in the light-induced calcium spike (n=8, FIG. 4M, MG vs. MG+PYR, two-tailed paired t test, p=0.046, t=2.42). In addition, the percentage of reactive fibers was reduced in cultures treated with MG#1 IgG and complement compared to controls (FIG. 4N, CTRL 78% vs. MG 32%, two-tailed unpaired t test, p<0.001, t=4.79). After application of PYR, we found a tendency towards a higher percentage of reactive fibers, which did not reach significance (FIG. 4N, MG vs. MG+PYR, two-tailed paired t test, p=0.065, t=2.19). Finally we sought to confirm the complement attack on the neuromuscular junction. For this purpose, cultures treated with MG#1 IgG and human complement as well as cultures treated with CTRL IgG and human complement for 24 h were stained with an antibody recognizing the human complement fragment C3c. Co-labeling with BTX and EYFP revealed targeted complement deposition onto muscle membranes in particular at the neuromuscular junction in MG but not in CTRL cultures. (FIG. 4O, P). Quantification revealed a significant deposition of complement at the neuromuscular junction in in MG cultures as compared to controls (FIG. 4Q, two-tailed unpaired t test, p=0.008, t=3.89). MN-only cultures did not show signs of toxicity when incubated with increasing amounts of MG and control IgG and complement.

### Discussion

Most anticipated applications of human spinal motorneurons in regenerative medicine (Davis-Dusenbery et al., 2014; Steinbeck and Studer, 2015) depend on their ability to functionally connect to skeletal muscle through neuromuscular junctions. However, quite generally the prospect of neuronal graft-to-host connectivity remains insufficiently validated due to technical limitations and the lack of suitable in vitro assays. Using optogenetics, we demonstrate for the first time that a human PSC-derived neuronal population with great therapeutic potential functionally connects to its bona fide human target tissue. In one study, murine embryonic stem cell-derived motorneurons under optogenetic control that were engrafted into partially denervated branches of the sciatic nerve of adult mice reinnervated lower hind-limb muscles (Bryson et al., 2014). However, the present study describes a fully human in vitro neuromuscular junction prepared from PSC-derived motorneuron and myoblast-derived muscle, wherein the motorneurons were able to form functional synapses with the muscle in vitro. Such an in vitro approach allows for an in depth functional characterization of neuromuscular connectivity and the clear exclusion of cell fusion in functional experiments. Without being bound by any particular theory, in our system neuromuscular synaptogenesis likely involves the secretion of agrin by MN terminals which signal through MuSK and rapsyn to induce the assembly of the neuromuscular junction (Sanes and Lichtman, 2001; Wu et al., 2010). The plaque-like AChR clustering (Marques et al., 2000) together with the stimulation-induced enhancement of contractility (FIG. 4J) suggest the formation of fully functional, yet still immature neuromuscular synapses.

Beyond the implications for human regenerative medicine, our novel culture system enables the modeling of neuromuscular disease in an all-human system. We show that specific functional and structural phenotypes of the classic neuromuscular disease myasthenia gravis and its treatment (Gold et al., 2008; Verschuuren et al., 2013) can be recapitulated in the neuromuscular co-cultures by the simple addition of myasthenia patient IgG and complement. Our findings indicate that both degenerative as well as regenerative aspects of neuromuscular disease can be studied in this human functional neuromuscular co-culture. Accordingly, we propose that the novel system may enable the dissection of disease processes originating from either side of the neuromuscular junction using patient specific iPSC derived neurons (Kiskinis et al., 2014) or muscle (Darabi et al., 2012; Skoglund et al., 2014).

### Methods

Synapsin-hChR2-EYFP hESC Line.

H9 human ES cells were transduced with lentiviral particles (pLenti-Syn-hChR2(H134R)-EYFP-WPRE) and plated at clonal density. Emerging colonies were screened by PCR for transgene integration and differentiated to assure stable long-term expression in all neuronal progeny.

### Generation of Spinal Motorneurons.

ES cells were plated in a confluent monolayer and neuralization was initiated by dual SMAD inhibition. For ventralization and caudalization purmorphamine and retinoic acid were added from day 1-15 (Calder et al., 2015). MN clusters emerging by day 20 were purified by sedimentation.

### Human Primary Myoblast Culture.

Human primary myoblasts were purchased from Life Technologies (adult donor) and Lonza (fetal donor). Both myoblast populations were grown in skeletal muscle growth medium (SkGM-2, Lonza). Differentiation was induced when myoblasts reached 70% confluence by exposure to media containing 2% horse serum.

### Initiation of Neuromuscular Co-Cultures.

Five to ten days after the initiation of myoblast differentiation, purified MN clusters were resuspended in matrigel plated centrally on top of the myocultures. Cultures were kept in MN differentiation media with the addition of 2% horse serum.

### Co-Culture Testing.

Maturing co-cultures were observed under 10× brightfield illumination while intermittently opening the shutter for fluorescent light (470 nm, 2mW/mm², approx. 300 ms pulse). Stably contracting regions were imaged under constant bright field illumination (40 ms exposure time, every 500 ms, 100 frames) in normal Tyrode's solution at room temperature. Optogenetic stimulation (470 or 630 nm, 2 mW/mm², approx. 300 ms pulse) was applied at indicated frequencies. For the quantification of movement multiple representative high contrast regions were automatically traced (MetaMorph Software).

### Calcium Imaging.

Myotubes or co-cultures on glass coverslips were incubated with the ratiometric calcium dye Fura-2 and imaged under continuous perfusion in normal Tyrode's solution. Myotubes were stimulated by acetylcholine. Co-cultures were illuminated for optogenetic stimulation for 10 ms at 470 nm (4 mW/mm2) every 5 s.

### Electrophysiology.

On matured EYFP+ MNs whole-cell current clamp recordings were performed at room temperature. Light-evoked APs were elicited using a 447 nm diode laser (OEM Laser) with light pulses of 5 ms at approximately 1 mW/mm2. Myotubes contracting in response to light stimulation were impaled with a sharp electrode for intracellular recordings.

Treatment with Human Serum and Myasthenia Gravis IgG Containing Antibodies to Nicotinic Acetylcholine Receptor.

Serum (containing complement) from 5 healthy donors was pooled and added to the media where indicated (2% v/v). IgG fractions were obtained from 2 severely affected MG patients, Sandoglobulin polyvalent IgG served as control (all 200 nM total IgG, patient #1 AChR antibody titer 576 nmol/l, #2 AChR antibody titer 17 nmol/l). Patients had given written consent to use their materials for research and this was approved by the Wurzburg University Medical School Ethics Committee.

### Immunocytochemistry and Imaging.

Cells or cultures were fixed in PFA and blocked with 5% FBS/0.3% Triton. Primary antibodies were incubated according to manufacturer recommendations followed by appropriate Alexa Flour-conjugated secondary antibodies. Stainings were either imaged on an inverted fluorescence microscope or confocal imaging was performed on an inverted Leica SP8 microscope equipped with white laser technology using a 40/63× oil immersion objective followed by subsequent data deconvolution where indicated.

### 7. REFERENCES

1. Amoroso, M. W., Croft, G. F., Williams, D. J., O'Keeffe, S., Carrasco, M. A., Davis, A. R., Roybon, L., Oakley, D. H., Maniatis, T., Henderson, C. E., et al. (2013). Accelerated high-yield generation of limb-innervating motor neurons from human stem cells. The Journal of neuroscience 33, 574-586.
2. Barker, A. T., Jalinous, R., and Freeston, I. L. (1985). Non-invasive magnetic stimulation of human motor cortex. Lancet 1, 1106-1107.
3. Boyden, E. S., Zhang, F., Bamberg, E., Nagel, G., and Deisseroth, K. (2005). Millisecond-timescale, genetically targeted optical control of neural activity. Nature neuroscience 8, 1263-1268.
4. Bryson, J. B., Machado, C. B., Crossley, M., Stevenson, D., Bros-Facer, V., Burrone, J., Greensmith, L., and Lieberam, I. (2014). Optical control of muscle function by transplantation of stem cell-derived motor neurons in mice. Science 344, 94-97.
5. Calder, E. L., Tchieu, J., Steinbeck, J. A., Tu, E., Keros, S., Ying, S. W., Jaiswal, M. K., Cornacchia, D., Goldstein, P. A., Tabar, V., et al. (2015). Retinoic Acid-Mediated Regulation of GLI3 Enables Efficient Motoneuron Derivation from Human ESCs in the Absence of Extrinsic SHH Activation. DOI:10.1523/JNEUROSCI.3046-14.2015
6. Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nature biotechnology 27, 275-280.
7. Chan, A., Lee, D. H., Linker, R., Mohr, A., Toyka, K. V., and Gold, R. (2007). Rescue therapy with anti-CD20 treatment in neuroimmunologic breakthrough disease. Journal of neurology 254, 1604-1606.
8. Cisterna, B. A., Cardozo, C., and Saez, J. C. (2014). Neuronal involvement in muscular atrophy. Frontiers in cellular neuroscience 8, 405.
9. Cunningham, M., Cho, J. H., Leung, A., Savvidis, G., Ahn, S., Moon, M., Lee, P. K., Han, J. J., Azimi, N., Kim, K. S., et al. (2014). hPSC-derived maturing GABAergic interneurons ameliorate seizures and abnormal behavior in epileptic mice. Cell stem cell 15, 559-573.
10. Darabi, R., Arpke, R. W., Irion, S., Dimos, J. T., Grskovic, M., Kyba, M., and Perlingeiro, R. C. (2012). Human ES- and iPS-derived myogenic progenitors restore DYSTROPHIN and improve contractility upon transplantation in dystrophic mice. Cell stem cell 10, 610-619.
11. Daube, J. R., and Rubin, D. I. (2009). Needle electromyography. Muscle & nerve 39, 244-270.
12. Davis-Dusenbery, B. N., Williams, L. A., Klim, J. R., and Eggan, K. (2014). How to make spinal motor neurons. Development 141, 491-501.
13. Espuny-Camacho, I., Michelsen, K. A., Gall, D., Linaro, D., Hasche, A., Bonnefont, J., Bali, C., Orduz, D., Bilheu, A., Herpoel, A., et al. (2013). Pyramidal neurons derived from human pluripotent stem cells integrate efficiently into mouse brain circuits in vivo. Neuron 77, 440-456.
14. Gold, R., Hohlfeld, R., and Toyka, K. V. (2008). Progress in the treatment of myasthenia gravis. Therapeutic advances in neurological disorders 1, 36-51.
15. Gouti, M., Tsakiridis, A., Wymeersch, F. J., Huang, Y., Kleinjung, J., Wilson, V., and Briscoe, J. (2014). In vitro generation of neuromesodermal progenitors reveals distinct roles for wnt signalling in the specification of spinal cord and paraxial mesoderm identity. PLoS biology 12, e1001937.
16. Kiskinis, E., Sandoe, J., Williams, L. A., Boulting, G. L., Moccia, R., Wainger, B. J., Han, S., Peng, T., Thams, S., Mikkilineni, S., et al. (2014). Pathways disrupted in human ALS motor neurons identified through genetic correction of mutant SOD1. Cell stem cell 14, 781-795.
17. Kuwabara, S., and Yuki, N. (2013). Axonal Guillain-Barre syndrome: concepts and controversies. The Lancet Neurology 12, 1180-1188.
18. Lancaster, M. A., and Knoblich, J. A. (2014). Organogenesis in a dish: modeling development and disease using organoid technologies. Science 345, 1247125.
19. Lee, G., Chambers, S. M., Tomishima, M. J., and Studer, L. (2010). Derivation of neural crest cells from human pluripotent stem cells. Nature protocols 5, 688-701.
20. Marques, M. J., Conchello, J. A., and Lichtman, J. W. (2000). From plaque to pretzel: fold formation and acetylcholine receptor loss at the developing neuromuscular junction. The Journal of neuroscience 20, 3663-3675.
21. Maury, Y., Come, J., Piskorowski, R. A., Salah-Mohellibi, N., Chevaleyre, V., Peschanski, M., Martinat, C., and Nedelec, S. (2015). Combinatorial analysis of developmental cues efficiently converts human pluripotent stem cells into multiple neuronal subtypes. Nature biotechnology 33, 89-96.
22. Mercuri, E., and Muntoni, F. (2013). Muscular dystrophies. Lancet 381, 845-860.
23. Moloney, E. B., de Winter, F., and Verhaagen, J. (2014). ALS as a distal axonopathy: molecular mechanisms affecting neuromuscular junction stability in the presymptomatic stages of the disease. Frontiers in neuroscience 8, 252.
24. Patani, R., Hollins, A. J., Wishart, T. M., Puddifoot, C. A., Alvarez, S., de Lera, A. R., Wyllie, D. J., Compston, D. A., Pedersen, R. A., Gillingwater, T. H., et al. (2011). Retinoid-independent motor neurogenesis from human embryonic stem cells reveals a medial columnar ground state. Nature communications 2, 214.
25. Philippidou, P., and Dasen, J. S. (2013). Hox genes: choreographers in neural development, architects of circuit organization. Neuron 80, 12-34.
26. Plomp, J. J., Morsch, M., Phillips, W. D., and Verschuuren, J. J. (2015). Electrophysiological analysis of neuromuscular synaptic function in myasthenia gravis patients and animal models. Experimental neurology DOI 10.1016/j.expneuro1.2015.01.007
27. Sahashi, K., Engel, A. G., Lambert, E. H., and Howard, F. M., Jr. (1980). Ultrastructural localization of the terminal and lytic ninth complement component (C9) at the motor end-plate in myasthenia gravis. Journal of neuropathology and experimental neurology 39, 160-172.
28. Sanes, J. R., and Lichtman, J. W. (2001). Induction, assembly, maturation and maintenance of a postsynaptic apparatus. Nature reviews Neuroscience 2, 791-805.
29. Sendtner, M. (2014). Motoneuron disease. Handbook of experimental pharmacology 220, 411-441.
30. Silva, N. A., Sousa, N., Reis, R. L., and Salgado, A. J. (2014). From basics to clinical: a comprehensive review on spinal cord injury. Progress in neurobiology 114, 25-57.
31. Skoglund, G., Laine, J., Darabi, R., Fournier, E., Perlingeiro, R., and Tabti, N. (2014). Physiological and ultrastructural features of human induced pluripotent and embryonic stem cell-derived skeletal myocytes in vitro. Proceedings of the National Academy of Sciences of the United States of America 111, 8275-8280.
32. Steinbeck, J. A., Choi, S. J., Mrejeru, A., Ganat, Y., Deisseroth, K., Sulzer, D., Mosharov, E. V., and Studer, L. (2015). Optogenetics enables functional analysis of human embryonic stem cell-derived grafts in a Parkinson's disease model. Nature biotechnology 33, 204-209.
33. Steinbeck, J. A., and Studer, L. (2015). Moving Stem Cells to the Clinic: Potential and Limitations for Brain Repair. Neuron 86, 187-206.
34. Titulaer, M. J., Lang, B., and Verschuuren, J. J. (2011). Lambert-Eaton myasthenic syndrome: from clinical characteristics to therapeutic strategies. The Lancet Neurology 10, 1098-1107.
35. Toyka, K. V., Drachman, D. B., Griffin, D. E., Pestronk, A., Winkelstein, J. A., Fishbeck, K. H., and Kao, I. (1977). Myasthenia gravis. Study of humoral immune mechanisms by passive transfer to mice. The New England Journal of Medicine 296, 125-131.
36. Verschuuren, J. J., Huijbers, M. G., Plomp, J. J., Niks, E. H., Molenaar, P. C., Martinez-Martinez, P., Gomez, A. M., De Baets, M. H., and Losen, M. (2013). Pathophysiology of myasthenia gravis with antibodies to the acetylcholine receptor, muscle-specific kinase and low-density lipoprotein receptor-related protein 4. Autoimmunity reviews 12, 918-923.
37. Wu, H., Xiong, W. C., and Mei, L. (2010). To build a synapse: signaling pathways in neuromuscular junction assembly. Development 137, 1017-1033.
38. Zhang, F., Vierock, J., Yizhar, O., Fenno, L. E., Tsunoda, S., Kianianmomeni, A., Prigge, M., Berndt, A., Cushman, J., Polle, J., et al. (2011). The microbial opsin family of optogenetic tools. Cell 147, 1446-1457.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A composition comprising an in vitro neuromuscular junction comprising a co-culture of a human motorneuron and a human skeletal muscle, wherein the motorneuron comprises a human pluripotent stem cell (PSC)-derived spinal motorneuron, and wherein the skeletal muscle comprises human myoblast-derived skeletal muscle or PSC-derived muscle.

2. The composition of claim 1, wherein the neuromuscular junction comprises PSC-derived muscle cells.

3. The composition of claim 1, wherein the motorneuron expresses detectable levels of one or more of homeobox gene 9 (HB9), neurofilament marker SMI32, Isletl (ISL1), homeobox transcription factor NKX6.1, oligodendrocyte transcription factor 2 (OLIG2), choline acetyltransferase (ChAT), acetylcholine esterase (ACHE), and agrin (AG).

4. The composition of claim 1, wherein the human PSC-derived spinal motorneuron is differentiated by contacting a human PSC with an effective amount of at least one Small Mothers Against Decapentaplegic (SMAD) inhibitor, at least one ventralizing factor, and at least one caudalizing factor.

5. The composition of claim 4, wherein the at least one SMAD inhibitor is selected from the group consisting of an inhibitor of Transforming growth factor β (TGFβ)/Activin-Nodal signaling and an inhibitor of bone morphogenetic proteins (BMP) signaling.

6. The composition of claim 5, wherein the inhibitor of TGFβ/Activin-Nodal signaling is SB431542.

7. The composition of claim 5, wherein the inhibitor of BMP signaling is LDN193189.

8. The composition of claim 4, wherein the at least one ventralizing factor comprises an activator of the hedgehog pathway.

9. The composition of claim 8, wherein the activator of the hedgehog pathway is selected from the group consisting of sonic hedgehog (SHH), purmorphamine, and combinations thereof.

10. The composition of claim 4, wherein the at least one caudalizing factor is selected from the group consisting of retinoic acid (RA), a Wingless (Wnt) activating factor, and combinations thereof.

11. The composition of claim 1, wherein the motorneuron expresses a light-sensitive protein.

12. The composition of claim 11, wherein the light-sensitive protein comprises a light-gated ion channel.

13. The composition of claim 12, wherein the light-gated ion channel is selected from the group consisting of rhodopsin, channelrhodopsin, halorhodopsin, archaerhodopsin, bacteriorhodopsin, proteorhodopsin, derivatives thereof, and combinations thereof.

14. The composition of claim 13, wherein the channelrhodopsin is channelrhodopsin-2.

15. The composition of claim 1, wherein the human motorneuron and human skeletal muscle are derived from cells isolated from a subject diagnosed with or at risk for having ALS, myasthenia gravis, or cachexia.

16. The composition of claim 1, wherein the human motorneuron and human skeletal muscle are co-cultured in the presence of immunoglobulin from a myasthenia gravis patient, and wherein the immunoglobulin comprises autoantibodies against proteins in the neuromuscular junction of the patient.

17. The composition of claim 1, wherein the human motorneuron and human skeletal muscle are co-cultured in the presence of blood, blood serum, and/or blood plasma from a subject diagnosed with, or at risk of having, cachexia.

18. The composition of claim 1, wherein the human motorneuron and human skeletal muscle are co-cultured in the presence of proteolysis factors and/or inflammatory cytokines.

19. The composition of claim 18, wherein the inflammatory cytokines are selected from the group consisting of tumor necrosis factor-alpha, interferon-gamma and interleukin-6.

20. A method for identifying an agonist of neuromuscular junction activity comprising stimulating the motorneuron of the in vitro neuromuscular junction according to claim 1, and contacting the neuromuscular junction with a candidate compound, wherein a candidate compound that increases the activity of the in vitro neuromuscular junction is selected as the agonist.

21. A method for identifying an agonist of neuromuscular junction activity comprising:
(a) stimulating the motorneuron of the in vitro neuromuscular junction according to claim 1 in the presence of a candidate compound, and determining the activity of the in vitro neuromuscular junction;
(b) stimulating the motorneuron of the in vitro neuromuscular junction according to claim 1 in the absence of the candidate compound, and determining the activity of the in vitro neuromuscular junction;
(c) comparing the activity in (a) and (b); and
(d) selecting the candidate compound as the agonist when the level of activity in (a) is greater than the level of activity in (b).

22. A method for identifying an antagonist of neuromuscular junction activity comprising stimulating the motorneuron of the in vitro neuromuscular junction according to claim 1, and contacting the neuromuscular junction with a candidate compound, wherein a candidate compound that decreases the activity of the in vitro neuromuscular junction is selected as the antagonist.

23. A method for identifying an antagonist of neuromuscular junction activity comprising:
(a) stimulating the motorneuron of the in vitro neuromuscular junction according to claim 1 in the presence of a candidate compound, and determining the activity of the in vitro neuromuscular junction;
(b) stimulating the motorneuron of the in vitro neuromuscular junction according to claim 1 in the absence of the candidate compound, and determining the activity of the in vitro neuromuscular junction;
(c) comparing the activity in (a) and (b); and
(d) selecting the candidate compound as an antagonist when the level of activity in (a) is less than the level of activity in (b).

24. A kit comprising the in vitro neuromuscular junction according to claim 1.

25. A kit comprising PSC-derived motorneurons and skeletal muscle, or co-cultures thereof.

26. A method of identifying genes that modulate neuromuscular junction activity comprising increasing or decreasing the level of expression of a gene in a motorneuron and/or muscle of a neuromuscular junction, and determining the activity of the neuromuscular junction, wherein an increase or decrease in neuromuscular junction activity that correlates with an increase or decrease in a gene's expression level indicates that the gene is a modulator of neuromuscular junction activity.

27. A method of preparing an in vitro neuromuscular junction comprising differentiating a pluripotent stem cell (PSC) into spinal motorneuron, and co-culturing the PSC-derived spinal motorneuron with skeletal muscle.
